(19) United States
(12) Patent Application Publication
(10) Pub. No.: US 2026/0317820 A1
(43) Pub. Date: Oct. 15, 2026

(54) METHODS AND SYSTEMS FOR RNA SEQUENCING AND ANALYSIS

(71) Applicant: THE UNIVERSITY OF CHICAGO, Chicago, IL (US)

(72) Inventors: Tao PAN, Chicago, IL (US); Christopher KATANSKI, Chicago, IL (US); Christopher P. WATKINS, Chicago, IL (US)

(73) Assignee: THE UNIVERSITY OF CHICAGO, Chicago, IL (US)

(21) Appl. No.: 19/116,480

(22) PCT Filed: Sep. 29, 2023
(86) PCT No.: PCT/US2023/075610

§ 371 Date: Mar. 28, 2025

Related U.S. Application Data
(60) Provisional application No. 63/411,271, filed on Sep. 29, 2022.

(51) Int. Cl. C12Q 1/6876; C12Q 1/6855; C12Q 1/6869

(52) U.S. Cl. CPC C12Q 1/6876; C12Q 1/6855; C12Q 1/6869; C12Q 2600/112; C12Q 2600/118; C12Q 2600/156; C12Q 2600/16

(57) ABSTRACT

Aspects of the present disclosure are directed to methods and systems for sample collection, tRNA sequencing, and diagnosis, prognosis, and treatment of viral infections such as SARS-CoV-2. Disclosed herein are methods for tRNA sequencing and analysis, including obtaining a tRNA profile of RNA from a nasopharyngeal sample. A tRNA profile may include abundance, fragmentation, and/or modification information for a plurality of tRNAs from a sample. Also disclosed are methods for diagnosis, prognosis, and/or treatment of a viral infection (e.g., SARS-CoV-2 infection) based on abundance, fragmentation, and/or modification information for one or more tRNAs from a subject having or suspected of having a viral infection.

### CROSS-REFERENCE TO RELATED APPLICATIONS

This application claims priority to and the benefit of U.S. Provisional Application No. 63/411,271 filed Sep. 29, 2022, the contents of which are incorporated into the present application by reference in its entirety.

### STATEMENT OF FEDERAL SUPPORT

This invention was made with government support under grant number AI141343 awarded by the National Institutes of Health. The government has certain rights in the invention.

### BACKGROUND

### I. Technical Field

Aspects of the present disclosure relate to at least the fields of molecular biology, virology, and immunology.

### II. Background

Emerging and re-emerging respiratory viruses can spread rapidly and cause pandemics as demonstrated by the recent severe acute respiratory syndrome coronavirus 2 (SARS-CoV-2). The early human responses to respiratory viruses are in the nasal cavity and nasopharyngeal regions. Defining biomarkers of disease trajectory at the time of a positive diagnostic test would be an important tool to facilitate decisions such as initiation of antiviral treatment.

A major response of biological systems to environmental change is regulated protein synthesis where transfer RNA (tRNA) plays a key role in decoding genetic information on-demand. tRNAs are small non-coding RNAs (74-95 residues) that read the genetic code and provide amino acids in protein synthesis. A human cell has several hundred tRNA sequences, up to 100 million tRNA transcripts, and each human tRNA contains on average 13 modifications (1,2). Modifications to tRNAs are dynamically regulated during cellular stress (3-5) and can affect decoding speed and accuracy of translation. Hypo-modified tRNAs can also become better substrates for RNase cleavage leading to tRNA fragment generation (6,7). tRNA fragments are a family of small RNAs that participate in many regulatory processes at the cellular and organismal levels (8-10). In the context of viral infections, tRNAs as well as tRNA fragments may facilitate viral replication (11,12).

Understanding the determinants of severity of viral infections is important for selecting the appropriate level of clinical care including initiation of therapeutic interventions aimed at preventing severe outcomes. In the case of emerging viral pathogens such as severe acute respiratory syndrome coronavirus-2 (SARS-CoV-2), a major challenge is the wide range of Coronavirus Disease 19 (COVID-19) severities. Many viral and host factors influence the symptom severity upon infection, including age, co-morbidities, host genetics and possibly SARS-CoV-2 variant. However, it remains difficult to predict which COVID-19 patients will develop mild or severe symptoms at the time of diagnosis.

Respiratory viruses such as SARS-CoV-2 are pathogens that can reach pandemic proportions. The early human responses to respiratory viruses are in the nasal cavity and nasopharyngeal regions. Defining biomarkers of disease trajectory at the time of a positive diagnostic test would be an important tool to facilitate decisions such as initiation of antiviral treatment.

### SUMMARY

Aspects of the present disclosure are based, at least in part, on the development of methods for sequencing and analysis of tRNAs from nasopharyngeal samples and on the discovery that distinct tRNA signatures (e.g., tRNA abundance, modification, and/or fragmentation) are associated with presence and/or severity of viral infections, including SARS-CoV-2 infection. Accordingly, certain aspects of the disclosure are directed to sample collection methods comprising isolating RNA from a nasopharyngeal sample from a subject having or suspected of having a viral infection and sequencing the RNA to obtain tRNA sequences. Also disclosed are viral treatment methods comprising administering a viral therapy to a subject following analysis of tRNA sequences from RNA isolated from a nasopharyngeal sample from a subject having or suspected of having a viral infection. Methods for diagnosis and prognosis of a viral infection are also disclosed.

Certain aspects concern a sample collection method comprising any one of the following steps: isolating RNA from a nasopharyngeal sample from a subject having or suspected of having a viral infection; assaying RNA; and sequencing the RNA to obtain tRNA sequences. In various aspects, one or more of the preceding steps is excluded. In various aspects, the RNA is isolated from a nasopharyngeal sample from a subject having or suspected of having a SARS-CoV-2 infection. In various aspects, assaying the RNA comprises performing template ligation qPCR on the RNA, including by one or more of the methods described herein. In some aspects, the sequencing step comprises obtaining a tRNA profile of the sample. The tRNA profile may comprise abundance, fragmentation, and modification information for at least 2 tRNAs, for at least 10 tRNAs, and/or for at least 100 tRNAs. In various aspects, the tRNA profile does not include one or more of abundance, fragmentation, or modification information. In some aspects, the sequencing step comprises multiplex small RNA sequencing. The tRNA sequences may comprise sequence of one or more tRNAs listed in Table 1 or Table 2. In various aspects, it is specifically contemplated that one or more tRNAs or tRNA modifications of Table 1 or Table 2 are excluded.

In some aspects, the tRNA sequences comprise sequences of one or more of the tRNA isoacceptor families AlaAGC, ArgACG, ProAGG, and GlnCTG. It is also contemplated that any of these isoacceptor families can be specifically excluded in various aspects. In some aspects, the method comprises determining a fragmentation profile of the one or more tRNA isoacceptor families. In certain aspects, the tRNA sequences comprise sequences of one or more of tRNAs chr1.trna32-MetiCAT, chr1.trna52-ProCGG, chr1.trna34-LeuCGG, and chr2.trna3-AlaAGC. It is also contemplated that any of these sequences can be specifically excluded in various aspects. In some aspects, the method comprises determining an abundance of the one or more tRNAs relative to ribosomal RNA in the sample. In some aspects, the tRNA sequences comprise tRNA modification information for one or more of tRNA modifications mtValTAC.m¹A9, chr1.trna54-LysTTT.m¹A58, chr1.trna59-GluCTC.m¹A57, and chr1.tRNA69-AspGTC.m¹A9. It is also contemplated that any of these modifications can be specifically excluded in various aspects. In some aspects, the method comprises determining a frequency of the one or more tRNA modifications. In some aspects, the method comprises determining a fragmentation profile of a tRNA of isoacceptor family ArgAGC, an abundance of tRNA chr1.tRNA32-MetiCAT, and modification information for tRNA modification mtValTAC.m¹A9. It is also contemplated that any of the preceding elements can be specifically excluded in various aspects.

In certain aspects, the viral infection is an influenza infection. The viral infection may be a coronavirus infection, which may be a SARS-CoV-2 infection. In some aspects, the method further comprises identifying the subject as having severe COVID-19 based on analysis of the tRNA sequences. In some aspects, the method further comprises identifying the subject as having mild COVID-19 based on analysis of the tRNA sequences. In some aspects, the method further comprises diagnosing the subject as having COVID-19. In some aspects, the method further comprises administering to the subject a COVID-19 therapy. The COVID-19 therapy may comprise an antibody therapeutic, an antiviral therapeutic, or convalescent plasma. It is also contemplated that any of these therapies can be specifically excluded in various aspects.

Certain aspects concern methods of treating a SARS-CoV-2 infection in a subject, the method comprising one or more of the following steps: isolating RNA from a nasopharyngeal sample from a subject having or suspected of having a SARS-CoV-2 infection; assaying RNA; sequencing the RNA to obtain tRNA sequences; and administering to the subject a therapeutically effective amount of a COVID-19 therapy. In various aspects, one or more of the preceding steps is specifically excluded. In various aspects, the RNA is isolated from a nasopharyngeal sample from a subject having or suspected of having a SARS-CoV-2 infection. In various aspects, assaying the RNA comprises performing template ligation qPCR on the RNA, including by one or more of the methods described herein. In certain aspects, the sequencing comprises obtaining a tRNA profile of the sample. The tRNA profile may comprise abundance, fragmentation, and modification information for at least 2 tRNAs, for at least 10 tRNAs, and/or for at least 100 tRNAs. In some aspects, the sequencing step comprises multiplex small RNA sequencing. The tRNA sequences may comprise sequence of one or more tRNAs listed in Table 1 or Table 2.

In some aspects, the tRNA sequences comprise sequences of one or more of the tRNA isoacceptor families AlaAGC, ArgACG, ProAGG, and GlnCTG. In some aspects, the method comprises determining a fragmentation profile of the one or more tRNA isoacceptor families. In certain aspects, the tRNA sequences comprise sequences of one or more of tRNAs chr1.trna32-MetiCAT, chr1.trna52-ProCGG, chr1.trna34-LeuCGG, and chr2.trna3-AlaAGC. In some aspects, the method comprises determining an abundance of the one or more tRNAs relative to ribosomal RNA in the sample. In some aspects, the tRNA sequences comprise tRNA modification information for one or more of tRNA modifications mtValTAC.m¹A9, chr1.trna54-LysTTT.m¹A58, chr1.trna59-GluCTC.m¹A57, and chr1.tRNA69-AspGTC.m¹A9. In some aspects, the method comprises determining a frequency of the one or more tRNA modifications. In some aspects, the method comprises determining a fragmentation profile of a tRNA of isoacceptor family ArgAGC, an abundance of tRNA chr1.tRNA32-MetiCAT, and modification information for tRNA modification mtValTAC.m¹A9. The COVID-19 therapy may comprise an antibody therapeutic, an antiviral therapeutic, or convalescent plasma. In some aspects determining an abundance of the one or more tRNAs relative to ribosomal RNA in the sample means assaying for the number of molecules of one or more tRNAs relative to ribosomal RNA (for example, by a ratio or by normalizing the amounts). It is also contemplated that any of the preceding elements can be specifically excluded in various aspects.

Certain aspects concern methods for diagnosing or prognosing a subject with COVID-19, the method comprising one or more of the following steps: isolating RNA from a nasopharyngeal sample from a subject having or suspected of having a SARS-CoV-2 infection; assaying RNA; sequencing the RNA to obtain tRNA sequences; and analyzing the tRNA sequences to identify the subject as having mild COVID-19 or severe COVID-19. In various aspects, one or more of the preceding steps is specifically excluded. In various aspects, the RNA is isolated from a nasopharyngeal sample from a subject having or suspected of having a SARS-CoV-2 infection. In various aspects, assaying the RNA comprises performing template ligation qPCR on the RNA, including by one or more of the methods described herein. In some aspects, the sequencing step comprises obtaining a tRNA profile of the sample. The tRNA profile may comprise abundance, fragmentation, and modification information for at least 2 tRNAs, for at least 10 tRNAs, and/or for at least 100 tRNAs. In some aspects, the sequencing step comprises multiplex small RNA sequencing. The tRNA sequences may comprise sequence of one or more tRNAs listed in Table 1 or Table 2. It is also contemplated that any of the preceding elements can be specifically excluded in various aspects.

In some aspects, the tRNA sequences comprise sequences of one or more of the tRNA isoacceptor families AlaAGC, ArgACG, ProAGG, and GlnCTG. In some aspects, the method comprises determining a fragmentation profile of the one or more tRNA isoacceptor families. In certain aspects, the tRNA sequences comprise sequences of one or more of tRNAs chr1.trna32-MetiCAT, chr1.trna52-ProCGG, chr1.trna34-LeuCGG, and chr2.trna3-AlaAGC. In some aspects, the method comprises determining an abundance of the one or more tRNAs relative to ribosomal RNA in the sample. In some aspects, the tRNA sequences comprise tRNA modification information for one or more of tRNA modifications mtValTAC.m¹A9, chr1.trna54-LysTTT.m¹A58, chr1.trna59-GluCTC.m¹A57, and chr1.tRNA69-AspGTC.m¹A9. In some aspects, the method comprises determining a frequency of the one or more tRNA modifications. In some aspects, the method comprises determining a fragmentation profile of a tRNA of isoacceptor family ArgAGC, an abundance of tRNA chr1.tRNA32-MetiCAT, and modification information for tRNA modification mtValTAC.m¹A9. It is also contemplated that any of the preceding elements can be specifically excluded in various aspects.

In some aspects, the subject is identified as having mild COVID-19. In some aspects, the subject is identified as having severe COVID-19.

Certain aspects concern methods for evaluating a subject having or suspected of having a SARS-CoV-2 infection, the method comprising one or more of the following steps: isolating RNA from a nasopharyngeal sample from the subject; assaying RNA; and sequencing the RNA to obtain a tRNA profile comprising abundance, fragmentation, and modification information for at least 2 tRNAs of Table 1 or Table 2. In various aspects, one or more of the preceding steps are specifically excluded. In various aspects, the RNA is isolated from a nasopharyngeal sample from a subject having or suspected of having a SARS-CoV-2 infection. In various aspects, assaying the RNA comprises performing template ligation qPCR on the RNA, including by one or more of the methods described herein. In some aspects, the tRNA profile comprises abundance, fragmentation, and modification information for at least 10 tRNAs of Table 1 or Table 2. In certain aspects, the sequencing comprises multiplex small RNA sequencing. In some aspects, the tRNA sequences comprise sequences of one or more of the tRNA isoacceptor families AlaAGC, ArgACG, ProAGG, and GlnCTG. In some aspects, the method comprises determining a fragmentation profile of the one or more tRNA isoacceptor families. In certain aspects, the tRNA sequences comprise sequences of one or more of tRNAs chr1.trna32-MetiCAT, chr1.trna52-ProCGG, chr1.trna34-LeuCGG, and chr2.trna3-AlaAGC. In some aspects, the method comprises determining an abundance of the one or more tRNAs relative to ribosomal RNA in the sample. In some aspects, the tRNA sequences comprise tRNA modification information for one or more of tRNA modifications mtValTAC.m¹A9, chr1.trna54-LysTTT.m¹A58, chr1.trna59-GluCTC.m¹A57, and chr1.tRNA69-AspGTC.m¹A9. In some aspects, the method comprises determining a frequency of the one or more tRNA modifications. In some aspects, the method comprises determining a fragmentation profile of a tRNA of isoacceptor family ArgAGC, an abundance of tRNA chr1.tRNA32-MetiCAT, and modification information for tRNA modification mtValTAC.m¹A9. It is also contemplated that any of the preceding elements can be specifically excluded in various aspects.

Because the SARS-CoV-2 virus causes COVID-19, any aspect discussed in the context of SARS-CoV-2 can be implemented with respect to COVID-19.

Further aspects of the present disclosure are based, at least in part, on the development of methods for detecting m¹A modifications through template ligation qPCR. Accordingly, certain aspects of the disclosure are directed to methods of quantitatively detecting N1-methyladenosine (m¹A) at a site of interest on a RNA, including but not limited to a tRNA, in a sample. In various aspects, the RNA is an mRNA, an miRNA, a tRNA, a lncRNA, or any other known RNA. It is also contemplated that the RNA is not an mRNA, an miRNA, a tRNA, a lncRNA, or any other known RNA in various aspects. Methods encompassing tRNAs are provided herein as an example but are not intended to limit the disclosed methods to detecting modifications on only tRNAs. In various aspects, the method comprises 1, 2, 3, 4, 5, 6 or more steps including any of the following: binding a first oligonucleotide to the tRNA in the sample; binding a second oligonucleotide to the tRNA in the sample; contacting the first oligonucleotide and second oligonucleotide with a ligase to generate a ligated product; assaying the sample to quantitatively detect an amount of ligated product; binding a third oligonucleotide and fourth oligonucleotide to a second site on the tRNA; contacting the third oligonucleotide and fourth oligonucleotide with a ligase to generate a second ligated product; and assaying the sample to quantitatively detect an amount of the second ligated product. It is specifically contemplated that any of the preceding steps can be excluded in various aspects.

In various aspects, quantitatively detecting the amount of ligated product and/or second ligated product, and/or quantitatively detecting m¹A means the product and/or m¹A is detected at a linear relationship (or other mathematically determinable relationship) relative to the abundance of the ligated product and/or m¹A present in a sample.

In various aspects, the first oligonucleotide is complementary to the tRNA both at the site of interest and upstream of the site of interest. For example the 5′ nucleotide of first oligonucleotide can be complementary to the site of interest and the remaining nucleotides can be complementary to the nucleotides that are towards the 5′ end of the tRNA. The first oligonucleotide need not be completely complementary to the tRNA. In various aspects, the first oligonucleotide is at least 80%, 85%, 90%, 95%, or 100%, or any range derivable therein, complementary to the tRNA. In various aspects, the first oligonucleotide is at least 80%, 85%, 90%, 95%, or 100%, or any range derivable therein, complementary to the sequence at and immediately upstream of the site of interest on the tRNA. In various aspects, the first oligonucleotide comprises a phosphorylated nucleotide, including a phosphorylated, thymine at its 5′-terminus. In various aspects, the second oligonucleotide is complementary to the tRNA directly downstream of the site of interest. For example, the second oligonucleotide can be complementary to the tRNA directly after the site of interest when reading the tRNA in a 5′ to 3′ direction. For example, the second oligonucleotide can be complementary to the nucleotides directly 3′ adjacent to the site of interest. The second oligonucleotide need not be completely complementary to the tRNA. In various aspects, the second oligonucleotide is at least 80%, 85%, 90%, 95%, or 100%, or any range derivable therein, complementary to the tRNA. In various aspects, the second oligonucleotide is at least 80%, 85%, 90%, 95%, or 100%, or any range derivable therein, complementary to the sequence immediately downstream of the site of interest on the tRNA. In various aspects, first oligonucleotide and second oligonucleotide are DNA oligonucleotides.

In various aspects, the ligase comprises a ligase capable of ligating an oligonucleotide comprising a 5′ phosphorylated nucleotide. In various aspects, the ligase comprises a SplintR ligase. In various aspects, the ligated product is not generated when the site of interest comprises an m¹A modification. For example, the ligated product is not generated when the site of interest comprises an m¹A modification due to the modification blocking the phosphorylated thymine being unable to bind to the methylated adenosine. In various aspects, the sample comprises a mixture of modified and unmodified adenosines at the site of interest. Accordingly, in various aspects, the amount of ligated product will correspond to the amount of RNAs comprising the unmodified adenosine relative to the total amount of RNA.

In various aspects, the first oligonucleotide is 10, 11, 12, 13, 14, 15, 16, 17, 18, 19, or 20 nucleotides in length. In various aspects, the first oligonucleotide is 15 nucleotides in length. In various aspects, the second oligonucleotide is 10, 11, 12, 13, 14, 15, 16, 17, 18, 19, or 20 nucleotides in length. In various aspects, the second oligonucleotide is 15 nucleotides in length.

In various aspects, the tRNA comprises at least 1 tRNAs of Table 1 or Table 2. In various aspects, the tRNA comprises a modification of Table 2. In various aspects, the site of interest comprises a modification of Table 2.

In various aspects, the second site where the third oligonucleotide and fourth oligonucleotide bind is different from the site of interest. In various aspects, the second site and does not comprise a known m¹A modification site. In various aspects, the third oligonucleotide binds directly adjacent to the fourth oligonucleotide. For example the second site may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleotides from the site of interest. The third oligonucleotides may bind directly upstream to the second site and the fourth oligonucleotide may bind directly downstream the second site. In various aspects, the method further comprises comparing the amount of the ligated product to the amount of the second ligated product.

In various aspects, the method comprises analyzing by quantitative PCR. For example, the amount of ligate product can be measured by qPCR. In various aspects, the amount of ligated product is measured by a qPCR probe that binds to the ligated product. In various aspects, the amount of the second ligated product is measured by a qPCR probe that binds to the second ligated product.

In various aspects, the sample is from any source, including any source described herein such as a nasopharyngeal sample. In various aspects, the sample comprises RNA isolated from a nasopharyngeal sample.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual,” “subject,” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is contemplated that any aspect discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIG. 1 shows tRNA fragmentation pattern among nasopharyngeal swap samples. Fractions of reads mapped to tRNA are shown for uninfected control (n=5), influenza infected (n=4), and SARS-CoV-2 infected (n=57) individuals.

FIGS. 2A-2B show that the fragmentation profile of specific tRNA isoacceptor families allows for separation of RNA obtained from nasopharyngeal swabs collected from SARS-CoV-2 infected individuals who developed mild or severe symptoms. 2A Uncorrected P-values for pairwise two-sided t-tests indicate several notable differences in fragmentation among specific tRNA isoacceptor families. 2B Fragmentation profiles for top anticodon-cleaved tRNAs are highlighted: tRNAAla(AGC), tRNAArg(ACG), tRNAPro(AGG), and tRNAGln(CTG). In addition to distinguishing among SARS-CoV-2 patients, fragmentation can provide diagnostic insight which compared to healthy patients. P-values: *<0.05; **<10-2; ***<10-3; ****<10-4. Two-sided t-tests with the number of samples passing a 10 read filter indicated on each plot.

FIGS. 3A-3B show that relative abundance of specific tRNA isodecoders to 5.8S rRNA in the same RNA samples allows for separation by infection status (uninfected, influenza and SARS-CoV-2 infected individuals). 3A Uncorrected P-values for pairwise two-sided t-tests indicate several notable differences in rRNA-normalized abundance among specific tRNA isodecoders. Analysis includes all sense-mapped reads for each isodecoder. Precise values are indicated in table S3. 3B rRNA-normalized abundance for notable tRNAs are highlighted: tRNAAla(AGC)c2t3, tRNAMet(CAT)c1t32, tRNALeu(CGG)c1t34, and tRNAPro(CGG)c1t52. In addition to distinguishing among SARS-CoV-2 patients, normalized abundance of tRNAPro(CGG)c1t52, among others, can provide diagnostic insight which compared to healthy patients. P-values: *<0.05; **<10-2; ***<10-3; ****<10-4. Two-sided t-tests with the number of samples passing a 10 read filter indicated on each plot.

FIGS. 4A-4B show that a specific tRNA modification profile in RNA obtained from nasopharyngeal swabs allows for separation of uninfected, influenza and SARS-CoV-2 infected individuals with mild or severe COVID-19 symptoms. 4A Uncorrected P-values for pairwise two-sided t-tests indicate differences among specific tRNA modifications. Modifications are detected as a ‘mutation’ derived from reverse transcriptase disincorporation when reading modified bases. Analysis includes sites with >2% mutation rate and >50 mapped reads. 4B Modifications exhibiting notable patterns are highlighted: m1A9 on tRNAAsp(GTC), m1A58 on tRNAGlu(CTC), m1A58 on tRNALys(TTT), m1A9 on mt-tRNAVal(TAC), and m1A57 on MetiCAT. P-values: *<0.05; **<10-2; ***<10-3; ****<10-4. Two-sided t-tests with the number of samples passing a 50 read filter indicated on each plot the 2% mutation rate filter was relaxed after individual sites were chosen.

FIGS. 5A-5D shows AUC curves of using tRNA abundance, modification, and fragmentation as biomarkers. Individual curves of fragmentation (tRNA^{Arg}(ACG), 5′tRF) (5A), abundance (tRNAiMet(CAT)) (5B), modification (mitochondrial tRNAVal, m¹A9) (5C) show AUC between 0.71-0.81. Combining these 3 generates an AUC of 0.99 (5D).

FIG. 6 shows methylation at cytosolic tRNAAsp(GTC) m¹A9. The modification fraction at position 9 for cytosolic tRNAAsp(GTC)c1t69 is plotted for each patient. Comparing between patient groups reveals that SARS-CoV-2 patients have reduced m¹A level compared to uninfected patients. Moreover, SARS-CoV-2 patients who develop severe symptoms have reduced methylation compared to those who develop mild symptoms. Wilcox test, *=0.05, **=0.01.

FIG. 7 shows global tRNA fragmentation pattern among nasopharyngeal swab samples. Comparison of global tRNA fragmentation between SARS-CoV-2 infected patients who go on to develop either mild or severe COVID19 symptoms. All tests are two-side t-test with the number of samples passing filters indicated on the plot. P-values: *<0.05; **<10-2; ***<10-3; ****<10-4.

FIG. 8 shows that relative abundance of global tRNA isodecoders to 5.8S rRNA in the same RNA samples allows for separation of patient groups. For each samples, all reads mapping to tRNA were summed, then divided by reads mapping to 5.8S rRNA. This ratio of measured global tRNA abundance to rRNA is different among patient groups, but not between SARS-CoV-2 patient groups. P-values: *<0.05; ns=no significance. Two-sided t-tests with the number of samples passing a 10 read filter indicated on the plot.

FIGS. 9A-9D shows the cross-validation scores of the samples for FIG. 5. The bar plots show the cross-validation scores of the samples in a logistic regression model of (9A) fragmentation (tRNAArg(ACG), 5′tRF), (9B) abundance (tRNAiMet(CAT)c1t32), (9C) modification (mitochondrial tRNAVal, m1A9) (9D) combination of the three biomarkers. Red, severe symptom samples. Blue, mild symptom samples.

FIGS. 10A-10D show TL-qPCR schematics and sensitivity with biological RNA. (10A) The structure of A-T match and potential m1A-T mismatch. The 5 linkers (5lkrT) with phosphorylated T at 5′-end can binds to complementary RNA template containing A or m1A at the exact position. SplintR ligase activates the ligation of the 5lkrT linker and the 3 linker at the joint position where the T matches to A of the RNA template, but not to m1A (indicated with italic Tx in red). Using ligated DNA of the 5lkrT as template in qPCR, a pair of primers can generate Cq signal with fluorescent probes, whereas no amplification for m1A. Comparing the difference of Cq value from A and m1A after normalization to an endogenous control, the proportion of m1A vs A in the RNA sample is obtained. (10B) Model tRNA results. Differential PCR cycles as a function of input RNA from 0.2-200 fmole for in vitro transcript of yeast tRNAPhe (left) and a synthetic human tRNAHis fragment (right). (10C) Detection of human tRFHis in HEK293T total RNA. The highest input amount is 50 ng total RNA which we determine to contain ~0.2 fmole of tRFHis by TL-qPCR. (10D) Detection of tRNAiMet in total HEK293T RNA. tRNAiMet is still detectable at 5 pg total RNA input.

FIGS. 11A-11F show TL-qPCR on human tRNAiMet mimicking RNA oligos. (11A) The oligos and linkers used in SplintR ligation for tRNA-iMet-58¹m-A. A-oligo and m¹A-oligo: two RNA oligos containing A or m¹A modified nucleotide as underlined. PF and PR: primer binding site during PCR. (11B) qPCR curves and (11C) the relative PCR product amounts of the four linkers 5lkrT/C/G/A in the templated SplintR ligation with the A oligo input at 0.1 nM concentration. (11D) Amplification curves and (11E) standard Cq curve of the dilution series of the A oligo input (#1 to #7 for 10× serial dilution from 1 nM to 1 fM) with H2O as a negative control (#8), generated with the linker 5lkrT in the SplintR ligation. (11F) The standard Cq curves of the ratio series of m1A/A oligo mixture input (#1 to #6) with final concentration at 1 nM of total RNA oligonucleotides in the SplintR ligation using the linker 5lkrT, with H2O as negative control.

FIGS. 12A-12E show the impact on the SplintR ligation efficiency by m¹A modification position and ligation temperature. (12A) Oligonucleotides used in D028A and DO29 m¹A assay and linker oligo design. (12B) linker oligos design at template site away from the m1A modification site. (12C) Amplification curves of linker 5lkrT/C/G/A in D028A and DO29m1A ligation. (12D) Amplification Cq plots and (12E) ΔCq values between m1A site and control site under different template splint ligation temperature (using 4 nt away oligos).

FIGS. 13A-13G show measuring m¹A58 modification in biological RNA. (13A) The sequences yeast tRNAPhe and oligonucleotides used in TL-qPCR for m¹A58 modification (red highlight). The two pair of linkers for yeast tRNAPhe-58m1A and for -5p are presented with Cy5 and TR (Texas Red), respectively as labeled probes separately. Cq values of Cy5 and TR probes of a set of 4× serial diluted inputs of yeast tRNAPhe samples from 0.8 pM to 3.1 fM without AlkB treatment (13B) and with AlkB treatment (13C). (13D) Differential Cq values between TR and Cy5 in the 4× serial diluted yeast tRNAPhe samples without and with AlkB treatment (remake this, y=ΔΔCq). (13E) Fraction of m¹A58 modification in yeast tRNAPhe (sample without AlkB treatment) measured by TL-qPCR. (13F) Oligonucleotides and linkers used in SplintR ligation for the m¹A58 of human tRNAiMet and tRNAHis, tRNATrp is used as an endogenous control. (13G) ΔCq values of 5lkrT and 5lkrT+4 on tRNAiMet (HEX labeled probe) and tRNAHis (TR-labeled probe) without (DM−) and with AlkB treatment (DM+) with FAM-labeled probe for tRNATrp as control.

FIGS. 14A-14B show the determination of the m¹A58 modification fraction of tRNAiMet and tRNAHis, in HEK293T cells. (14A) m¹A58 modification fraction detected by TL-qPCR for tRNAiMet was 70.3±2.8% and for tRNAHis 95.3±1.2%. Average of n=3 biological replicates. (14B) Modification index (MI) around the m¹A58 (±5 nt) site measured by DM-tRNA-seq for tRNAiMet and tRNAHis. The m¹A58 modification fractions of tRNAiMet and tRNAHis were estimated to be 77% and 95%, respectively.

FIGS. 15A-15B show analysis of the RNA templated ligation on synthesized RNA A and m1A oligonucleotides in FIG. 2. (15A) 10 pmol of total RNA oligo serial mixtures (lane 1 to 6) consisting of m1A and A for tRNA-iMet and with 10 μM of each 5′-lkrT and 3′-lkr for tRNA-iMet. Lane 1N: 100% input of m1A oligo without SplintR ligase. Lane 7: Water as negative control (revised this with quantitation, no need to show the actual gel). (15B). ESI-MS analysis of the m1A-containing oligo.

FIGS. 16A-16C show analysis of the RNA templated ligation on synthesized RNA A and m1A oligonucleotides in FIG. 3. (16A) 10 pmol of total RNA oligo serial mixtures (lane 1 to 6) consisting of m1A and A for tRNA-iMet and with 10 μM of each 5′-lkrT and 3′-lkr for tRNA-iMet. Lane 1N: 100% input of m1A oligo without SplintR ligase. Lane 7: Water as negative control (revised this with quantitation, no need to show the actual gel). (16B) Relative TL-qPCR product amount of 5′ linker oligos with T, C, G, or A at 5′ end. T-ending linker oligo with unmodified oligo was used as the control. The relative product amounts of other samples were too low to be visible in the figure. (16C) Relative efficiency of linker pairs for D028-m1A ligation at 32C/37C/42C/47C. TL-qPCR product amount at position T9 and 37° C. was used as the control (100%).

### DETAILED DESCRIPTION

Activated immune cells known as granulocytes secrete RNases as part of the innate immune response. In humans, eight secreted RNases have been identified, each with a range of antiviral, antibacterial and/or cytotoxic functions (13,14). Without wishing to be bound by theory, it is understood that tRNAs, due to their high abundance and RNase resistance, represent one of the best opportunities to detect early immune activation in the context of viral infection by measuring their ongoing fragmentation, alteration in abundance and in modification levels. These measurements can be made directly from virally lysed or apoptotic epithelial tissues, which often represent the sites of initial respiratory viral infection.

Here, in certain aspects, Multiplex Small RNA-seq (MSR-seq, (15)) is applied to identify tRNA-based biomarkers to predict viral infection (e.g., SARS-CoV-2) severity at the time of a positive test. MSR-seq simultaneously measures tRNA abundance, fragmentation and modifications in a single sequencing library generated directly from RNA isolated from the viral transport media of the diagnostic nasopharyngeal swab. As described herein, multiple tRNA properties vary significantly among individuals with COVID-19 who develop no/mild versus severe symptoms, thus indicating the biomarker potential of human nasal tRNA responses to an infectious disease such as COVID-19.

### I. Sample Analysis

Aspects of the present disclosure are directed to methods for diagnosis and prognosis of infections, including coronavirus infections such as SARS-CoV-2 infection. As disclosed herein, collection and analysis of tRNA sequences from a subject having, or suspected of having, an infection is, in some aspects, useful in infection diagnosis and/or identifying infectious disease severity. In some aspects, disclosed are methods for diagnosing a subject as having a viral infection (e.g., a SARS-CoV-2 infection). In some aspects, disclosed are methods for identifying a subject as having a mild viral infection (e.g., mild COVID-19). In some aspects, disclosed are methods for identifying a subject as having a severe viral infection (e.g., severe COVID-19). Such methods may also comprise making a treatment decision, providing a treatment recommendation, and/or providing an antiviral therapy to a subject based on the analysis of the tRNA sequences from the subject.

In some aspects, a method of the disclosure comprises obtaining a nasopharyngeal sample from a subject having or suspected of having a viral infection. Various methods for collection and storage of nasopharyngeal samples are recognized in the art and contemplated herein. The method may further comprise isolating one or more biomolecules from the sample. In some aspects, the method comprises isolating nucleic acid (e.g., RNA, DNA) from the nasopharyngeal sample. In particular aspects, the method comprises isolating RNA from the sample. The RNA may be or comprise one or more types of “small RNA,” such as tRNA, snRNA, micro-RNA, and tRNA fragments. In some aspects, the RNA comprises tRNA.

In certain aspects, the disclosed methods comprise sequencing RNA obtained or isolated from a biological sample (e.g., nasopharyngeal sample) to obtain sequences of one or more types of small RNA. Sequencing RNA may comprise multiplex small RNA sequencing (MSR-seq) to obtain tRNA sequences. tRNA sequences obtained from a sample may be analyzed to generate a tRNA profile of the sample. As used herein, a “tRNA profile” describes a set of information related to a plurality of tRNAs from a biological sample. A tRNA profile may include one or more of abundance, fragmentation, and modification information for at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 different tRNAs from a biological sample, or more. In some aspects, a tRNA profile is a set of abundance, fragmentation, and modification information for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 different tRNAs from a biological sample. In some aspects, tRNA abundance describes an amount of a particular type of tRNA relative to a control such as, for example, 5.8S rRNA. In some aspects, tRNA fragmentation describes the extent of tRNA fragmentation in a sample. tRNA fragmentation information may be represented by, in some cases, a “fragmentation index,” describing a value determined based on comparing sequence coverage from the 5′ half of a tRNA transcript to that of the 3′ half of the same tRNA transcript. In some aspects, tRNA modification describes a presence and/or frequency of a particular tRNA modification (e.g., methylation at a particular residue).

The disclosed methods may, in some cases, further comprise diagnosis, prognosis, and/or treatment of a viral infection in a subject. Thus, in some aspects, disclosed are methods comprising diagnosing a viral infection (e.g., SARS-CoV-2 infection, influenza infection, etc.) in a subject based on sequencing and analysis of tRNA (e.g., a tRNA profile) from a subject suspected of having a viral infection. In some aspects, disclosed are methods comprising prognosis of a viral infection, for example identifying a subject as having a mild or severe viral infection, based on sequencing and analysis of tRNA (e.g., a tRNA profile) from a subject having a viral infection. Also disclosed are methods for treatment of a viral infection, where a particular treatment is selected based on sequencing and analysis of tRNA (e.g., a tRNA profile) from a subject having a viral infection. Thus, in one example, where analysis of a tRNA profile from a subject indicates that the subject has a severe viral infection (e.g., severe COVID-19), the method comprises treating the subject with an antiviral therapy (e.g., COVID-19 therapy). Antiviral therapies contemplated herein include any therapy that treats, prevents, and/or improves or alleviates the symptoms of one or more viral infections, including a SARS-CoV-2 infection. Certain antiviral therapies are known in the art and contemplated herein, examples of which include remdesivir, COVID-19 convalescent plasma, and anti-SARS-CoV-2 spike protein antibodies (e.g., bamlanivimab).

As described herein, abundance, fragmentation, and/or modification information of one or more tRNAs and/or tRNA isoacceptor families can be used to uniquely identify the presence or absence of a particular viral infection or to identify whether a subject has a mild or severe viral infection (e.g., mild or severe COVID-19). In some aspects, the disclosed methods and systems comprise sequencing RNA from a sample containing at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 of the tRNAs of Table 1 or Table 2, or fragments thereof, or any tRNA in the isoacceptor families of Table 3. Abundance, fragmentation, and/or modification information may be obtained for the one or more tRNAs, thereby generating a tRNA profile.

TABLE 1
Significant tRNAs

| tRNA | ID (hg19) |
|---|---|
| tRNA^{Ala}(AGC) | chr2.trna3-AlaAGC |
| tRNA^{Pro}(CGG) | chr1.trna52-ProCGG |
| tRNA^{iMet}(CAT) | chr1.trna32-MetiCAT |
| tRNA^{Leu}(CAG) | chr1.trna34-LeuCAG |

TABLE 2
Significant tRNA modifications

| tRNA | Modification |
|---|---|
| mtValTAC | m1A9 |
| chr1.tRNA69-AspGTC | m1A9 |
| chr1.trna59-GluCTC | m1A57 |
| chr1.trna54-LysTTT | m1A58 |

TABLE 3
Significant tRNA Isoacceptor Families
tRNA Family

tRNA^{Ala}(AGC)
tRNA^{Arg}(ACG)
tRNA^{Pro}(AGG)
tRNA^{Gln}(CTG)

### II. Viruses

Aspects of the present disclosure relate to treatment, analysis, or use of a virus, a sample comprising a virus, a sample suspected of comprising a virus, or a sample from a subject having or suspected of having a viral infection. In some aspects, the disclosed methods include diagnosis, prognosis, treatment and/or prevention of a viral infection. In some aspects, disclosed are compositions comprising one or more antiviral agents. In some aspects, disclosed are methods for diagnosis of a viral infection. In some aspects, disclosed are methods for detection of a virus in a sample.

### A. Coronaviruses

In particular aspects, the virus is from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and about 40 species. They are enveloped viruses having a positive-sense single-stranded RNA genome and a nucleocapsid having helical symmetry. The genome size of coronaviruses ranges from about 26-32 kilobases.

The present disclosure encompasses treatment or prevention of infection of any virus in the Coronaviridae family. In certain aspects, the disclosure encompasses treatment or prevention of infection of any virus in the subfamily Coronavirinae and including the four genera, Alpha-, Beta-, Gamma-, and Deltacoronavirus. In specific aspects, the disclosure encompasses treatment or prevention of infection of any virus in the genus of Betacoronavirus, including the subgenus Sarbecovirus and including the species of severe acute respiratory syndrome-related coronavirus. In specific aspects, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of severe acute respiratory syndrome-related coronavirus, including at least SARS-CoV-2. In specific aspects, the virus has a genome length between about 29000 to about 30000, between about 29100 and 29900, between about 29200 and 29900, between about 29300 and 29900, between about 29400 and 29900, between about 29500 and 29900, between about 29600 and 29900, between about 29700 and 29900, between about 29800 and 29900, or between about 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular aspects, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular aspects, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses.

### III. Sample Collection and Preparation

In certain aspects, methods involve obtaining a sample (also “biological sample”) from a subject. The sample may be obtained from any other source including but not limited to blood, sweat, hair follicle, buccal tissue, tears, mucus, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be a cell-free sample (e.g., serum, plasma). The biological sample may be obtained using any method known to the art that can provide a sample suitable for the analytical methods described herein. The sample may be obtained by non-invasive methods including but not limited to: scraping of the skin or cervix, swabbing of the cheek, nasopharyngeal swab, saliva collection, urine collection, feces collection, or collection of menses, tears, or semen. In certain aspects of the present disclosure, a biological sample is a nasopharyngeal sample (i.e., a sample obtained from a nasopharyngeal swab of a subject).

The sample may be a sample comprising cell-free nucleic acid. Cell-free nucleic acid includes, for example, cell-free DNA (cfDNA) and cell-free RNA (cfRNA). Cell-free nucleic acid may be isolated, extracted, or otherwise purified from a biological sample for further analysis or processing using the methods and compositions disclosed herein. In some aspects, a sample comprises at least, at most, or about 1000, 900, 800, 700, 600, 500, 400, 300, 200, 100, 50, 40, 30, 20, 10, 5, 4, or 3 ng of nucleic acid, or any range or value derivable therein.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple tissue samples may be obtained for diagnosis by the methods described herein. In other cases, multiple samples, such as one or more samples from one tissue type and one or more samples from another specimen may be obtained for diagnosis by the methods. In some cases, multiple samples such as one or more samples from one tissue type and one or more samples from another specimen may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a nasopharyngeal sample, a urine sample, a fecal sample, a buccal sample, or a saliva sample.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects, the subject may be referred to a specialist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

A sample collection of the present disclosure may comprise, subsequent to obtaining a biological sample from a subject, isolating one or more biomolecules (e.g., protein, nucleic acid) from the sample. In some aspects, a sample collection method disclosed herein comprises isolating nucleic acid (e.g., DNA, RNA) from a biological sample from a subject. In certain aspects, the biological sample is a nasopharyngeal sample.

### IV. Sequencing

Aspects of the present disclosure include processing and analysis of nucleic acid from a biological sample (e.g., a nasopharyngeal sample). In some aspects, the methods of the disclosure include a sequencing method. Various sequencing methods are recognized in the art and contemplated herein. In some aspects, a method of the present disclosure comprises a particular sequencing method (e.g., MSR-seq).

### A. DNA Sequencing

In some aspects, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some aspects, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage.

### B. RNA Sequencing

In some aspects, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some aspects, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer.

a. MSR-seq

One example method for RNA sequencing which may be used in certain aspects of the disclosure is multiplex small RNA sequencing (MSR-seq). MSR-seq is an RNA sequencing platform based on the design of a biotinylated oligonucleotide that is used for barcode adapter ligation, immobilization, on-bead reverse transcription, second adapter ligation, and PCR. MSR-seq is a technique optimized for sequencing of small RNAs, including tRNA, snRNA, micro-RNA, and tRNA fragments. MSR-seq allows for, and in some cases comprises, enzymatic and chemical treatment of RNA on-bead, enabling investigation of RNA modifications and other applications. MSR-seq is described in Watkins, C. P., et al. (2022) Nature Communications, 13, 2491 and U.S. Patent Application [[PCT/US21/58258]], each of which is incorporated herein by reference in its entirety. In some aspects, MSR-seq is used to obtain tRNA sequences from a sample.

### EXAMPLES

The following examples are included to demonstrate certain aspects of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific aspects which are disclosed and still obtain a like or similar result without departing from the spirit and scope of the invention.

### Example 1—Analysis of tRNA Abundance, Modification and Fragmentation in Nasopharyngeal Swaps as Markers of COVID-19 Severity

Multiplex Small RNA-seq (MSR-seq, (15)) was applied to identify tRNA-based biomarkers to predict SARS-CoV-2 infection severity at the time of a positive test. MSR-seq simultaneously measured tRNA abundance, fragmentation and modifications in a single sequencing library generated directly from RNA isolated from the viral transport media of the diagnostic nasopharyngeal swab. The results showed that multiple tRNA properties vary significantly among individuals with COVID-19 who develop no/mild versus severe symptoms, thus indicating the biomarker potential of human nasal tRNA responses to an infectious disease such as COVID-19 or other viral infection.

### Materials and Methods

### Ethics Statement

The Mount Sinai Pathogens Surveillance Program (MS-PSP) systematically collects residual nasopharyngeal swab samples that tested positive for respiratory pathogens (e.g., SARS-CoV-2, Influenza A virus) after clinical testing is completed. A subset of representative specimen is selected for viral genome sequencing in the context of precision surveillance (16,17). The Institutional Review Board of the Icahn School of Medicine at Mount Sinai reviewed and approved MS-PSP as well as the disclosed studies.

Total RNA Extraction from Viral Transport Media

Total RNA was extracted from viral transport media (VTM) of the nasopharyngeal swabs using high-throughput specimen processing (KingFisher™ Flex Purification System, ThermoFisher, cat. 5400610). The MagMax™ mirVana™ Total RNA Isolation Kit (ThermoFisher, cat. A27828) was used to extract total RNA from 250 μL of viral transport medium, as per the manufacturer's protocol. The total nucleic acid concentration measured by nanodrop absorbance ranged from 4-40 ng/μL.

### RNA Sample Selection

Selected for study were 56 RNA samples extracted from nasopharyngeal swabs that tested positive for SARS-CoV-2, four RNA samples from nasopharyngeal swabs that tested positive for Influenza A, and five RNA samples from individuals without any known viral infection (uninfected control). All SARS-CoV-2 tested samples were collected during the first wave of the SARS-CoV-2 pandemic in New York City (March-May 2020). Thirty-five RNA samples were extracted from VTM of nasopharyngeal swabs collected from individuals who tested positive for SARS-CoV-2 but did not require hospitalization at the time of testing (Group I) while 21 RNA samples were extracted from VTM of nasopharyngeal swabs collected from individuals who were admitted to the hospital due to severe COVID-19 manifestations at the time of testing (Group II).

### Multiplex Small RNA Sequencing Library Construction

MSR-seq library construction followed the same procedure as described previously (15). A total of 10 μL of each extracted sample described above were used for sequencing library construction. The key features of MSR-seq are to first ligate total RNA with a bar-coded capture hairpin oligonucleotide which enables pooling of up to 12 bar-coded samples for all subsequent steps. The specific design of the capture hairpin oligo allows for all subsequent steps to be carried out on magnetic streptavidin beads. The basic steps of library construction include (i) deacylation to remove charged amino acids to tRNA; (ii) 3′ end repair to remove all 3′ phosphate/cyclic phosphate; (iii) first ligation of the barcoded hairpin oligonucleotide; (iv) sample pooling, mix with streptavidin beads, wash; (v) remove 3′ phosphate of the capture hairpin oligo; wash; (vi) reverse transcription using a thermophilic RT and overnight extension; (vii) remove RNA; (viii) periodate oxidation to block unligated hairpin oligonucleotide; (ix) second ligation of PCR primer; (x) PCR using Illumina® index primers.

For low abundant RNA samples, a gel purification step of the final PCR products was added to remove the excess primer only products as follows. After PCR, amplicons were concentrated using the Zymo DNA Clean & Concentrate spin columns (7 equivalents of DNA binding buffer to 1 equivalent of PCR reaction; e.g. 350 μL of DNA binding buffer to 50 μL PCR reaction). Samples were eluted in 12 μL of deionized, autoclaved water.

After eluting off the column, samples were mixed with 2.5 μL of 6× TriTrack gel loading dye and loaded onto a 6% Novex TBE gel. Samples, together with dsDNA size markers, were electrophoresed at 180 volts for approximately 40 minutes. After electrophoresis, the gel was incubated for 10 minutes in 1×SYBR Gold and then imaged on a blue light box. Samples were cut from approximately 170 to 300 bp, whereas the primer alone products containing barcodes and indexes were approximately 140-145 bp.

After cutting, gel fragments were crushed using a 1 mL pipette tip and then 500 μL of gel elution buffer (200 mM KCl, 50 mM KOAc, pH 7). Gel fragments were incubated overnight (12+ hours). After overnight incubation, samples were centrifuged for 10 minutes at room temperature at 10,000 g⁻¹. The supernatant was then collected and the samples were then centrifuged again to remove any remaining gel fragments. Next, 1 μL of GlycoBlue and 500 μL of isopropanol were added to each tube and the tubes were then placed in a −80° C. freezer for a minimum of 1 hour.

After cold incubation, the samples were centrifuged at 4° C. for 1 hour at 17,000 g⁻¹. The supernatant was then removed and the remaining pellet was resuspended in 20 μL of deionized, autoclaved water.

### Data Analysis

Read processing and mapping: Libraries were sequenced on Illumina® Nova-seq S1-200 platform, 100 bp paired-end. First, paired end reads were split by barcode sequence using Je demultiplex with options BPOS=BOTH BM=READ_1 LEN=4:6 FORCE=true C=false 6. Next read 2 files were used to map with bowtie2 with the following parameters: −q −p 10—local no-unal. Reads were mapped to curated hg19 list of non-redundant tRNA genes with tRNAScan score>40 (18). Bowtie2 output sam files were converted to bam files, then sorted using samtools (19). Next IGV was used to collapse reads into 1 nt window. IGV output .wig files were reformatted using custom python scripts (available on GitHub at github.com/ckatanski/MSR-seq). The bowtie2 output Sam files were also used as input for a custom python script using PySam, a python wrapper for SAMTools (github.com/pysam-developers/pysam) to sum all reads that mapped to each gene. Data was visualized with custom R scripts (available on GitHub at github.com/ckatanski/CovidNasalSwabs_2022ck).

Total mapped reads to tRNA and 5.8S/5S rRNA were obtained ranging from 0.18-13 million with mapping percentages ranging from 2.5-16.1%.

tRNA fragmentation: tRNA fragments were identified as previously described (15). Briefly, the precise 3′ end of the read in the MSR-seq procedure represents the 3′ end of the RNA present in the sample. The inventors binned the ends with 3′ ends mapped to the individual tRNA genes between nucleotides 20-30, 30-40, 40-50, 50-60 and >60 (full-length). These bins roughly correspond to the stem loops in tRNA structure and can be used to characterize the broad types of tRNA fragment. The fraction of a fragment was calculated by comparing the number of reads in one bin compared to all the reads in every bin for a particular gene from a particular patient. To summarize the data, the number of reads that sort to each bin among all genes was summed for each patient.

Fragmentation index: To determine the extent of tRNA fragmentation, a ‘fragmentation index’ was calculated comparing the median coverage from the 5′ half of the transcript (bases 10-30) to the median coverage of the 3′ half (40-60). This index is affected by readthrough of modifications, but it is useful for between patient comparisons. To condense tRNA isodecoder data into isoacceptor families, the median values of coverage were summed. Further calculation was restricted to families where the median 5′ and 3′ coverage was greater than 10 reads. Fragmentation index was then calculated as the ratio of these 5′ sums and 3′ sums. Fragmentation index of each isoacceptor family is compared for each SARS-CoV-2 patient.

tRNA abundance: Relative abundance of individual tRNA isodecoders was normalized to the 5.8S rRNA reads within each sample. This normalized abundance was then compared between patient groups.

tRNA modification: Mutation rates from bowtie mapping at individual sites were used to estimate modification rates. Analysis focused on well characterized sites with known modifications. Mutation rate is not a 1-to-1 output for modification fraction, but it is known to vary linearly. Thus relative changes are a reliable metric for relative changes in modification levels. Analysis was limited to sites and samples with >50 reads and a >2% mutation rate. After initial site selection, the 2% mutation filter was relaxed for individual site analysis so as to include samples which may have been excluded from initial screening.

P-value calculations: To identify differentially expressed, fragmented, or modified tRNAs, patient group comparisons for individual genes were performed with pairwise two-sided t-tests with no correction. To normalize tRNA abundance reads among patients, all abundances were normalized to the well detected 5.8S rRNA reads within the same sample. This normalized abundance was calculated for every tRNA gene using all sense-mapped reads. Comparing abundance of an individual gene between patient groups was done with a two-sided pairwise t-test with no correction. Analysis was restricted to isodecoders and samples with >10 reads for the respective tRNA sequence. To compare tRNA fragmentations, reads were subdivided based on the position of the 3′ end mapping. With MSR-seq, this is a faithful representation of the biological 3′ end of the tRNA and can separate fragments from full length tRNAs. Calculation was restricted to only sense reads, and fragments with >10 reads. Notably, similar analysis cannot be done for the 5′ end since truncated 5′ ends could reflect biological fragments or premature termination of reverse transcription in sequencing library preparation. To compare tRNA modifications among patient groups, the mutation rate at every base for every gene was calculated between patient groups using a two-sided pairwise t-test with no correction at a filter of >50 reads per site.

Logistic regression (LR) models and ROC curves: The samples with non-NULL values in the selected features were used to build the LR models (20-22). The data were shuffled and normalized. Then, 3-fold cross-validation was performed with 1 or 3 selected features and the scores of each sample are predicted. Specificity, sensitivity and AUC (area under curve) were calculated (20) and the ROC curves drawn (23) with python. The combine metric is a linear combination of either: abundance (rpm/5.8S rpm) of tRNAiMet-c1t32; mutation rate at position 9 for mitochondrial tRNAVal; fragmentation calculated with tRNAAla(AGC) isoacceptor family in the 30-40 bin; and abundance of tRNATrp-c17t12 (rpm/5.8S rpm) or abundance (rpm/5.8S rpm) of tRNA^{iMet}-c1t32; mutation rate at position 9 for mitochondrial tRNA^{Val}; fragmentation calculated with tRNA^{Arg}(ACG) isoacceptor family in the 30-40 bin. These relative weights for each of these metrics were optimized using “maxadjAUC” package for R (24). A linear combination of these metrics was combined into a single scoring parameter for the combine ROC.

### Results

The tRNA profile (abundance, modification and fragmentation) was analyzed using total RNA extracted from residual diagnostic specimen collected from individuals with symptoms suggestive of upper respiratory tract infection who tested positive for SARS-CoV-2. These samples were collected during the first wave of the pandemic when only ancestral SARS-CoV-2 variants were circulating. It was demonstrated that high quality small RNA sequencing data can be obtained from viral transport media of nasopharyngeal swabs used for diagnostic nucleic acid amplification testing (NAAT). These biospecimen are of low-biomass and contain only very small amounts of RNA. Using a new library construction technology for tRNA sequencing that uses total RNA from any biological source and on-bead library construction (15), tRNA-seq data was obtained informing simultaneously on full-length tRNA abundance, certain tRNA modifications, as well as 5′ tRNA fragments (5′tRF). After library construction and sequencing RNAs were found that could be mapped to tRNA and other small RNAs at appreciable rates with average mapped read counts of 1,006,000, 131,000, 1,102,500 for all small RNA and 397,500, 77,100, 285,000 for tRNA of the uninfected, influenza, and SARS-CoV-2 infected samples, respectively. While these rates are lower than tRNA-seq from cell cultures, they still reveal the potential to obtain high quality small RNA sequencing data from viral transport media of nasopharyngeal swabs used for diagnostic nucleic acid amplification testing (NAAT). Ultimately RNA-seq would be too costly and time-consuming to serve as a practical tool for triage, these studies provide for identification of potential prognostic biomarkers for further development using scalable nucleic acid technologies such as RT-qPCR.

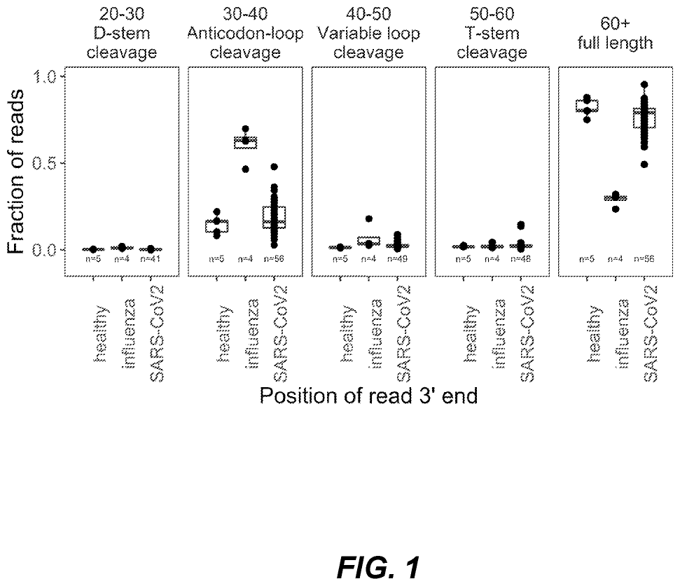

The inventors first analyzed 5′tRF differences among the SARS-CoV-2, influenza, and uninfected groups by binning the tRNA reads with the 3′ end in different tRNA positions ((15), FIGS. 1 and 8A). Approximately 85% of the tRNA reads had 3′ ends past nucleotide 60 which roughly corresponded to the full-length tRNAs in the analysis. tRNA fragmentation occurred extensively in all samples, compared to well-controlled samples from cell culture (15). Globally, fragmentations of tRNAs were present with cleavage sites in all tRNA regions. As expected, cleavage in the anticodon loop region (3′ ends within position 30-40 of tRNA, anticodon positions approximately 34-36) generated the highest amount of tRF products. Since fragmentation was not evenly distributed among accessible loop regions, this suggests some level of biological specificity. Influenza-infected patients showed a much greater degree of fragmentation compared to healthy subjects, though only 4 samples were available for analysis, limiting the strength of this observation. SARS-CoV-2 infected patients also showed greater fragmentation compared to healthy subjects. Interestingly, SARS-CoV-2 patients who developed mild symptoms showed a significantly higher degree of global tRNA fragmentation compared to patients who developed severe symptoms (FIG. 8B). This could reflect the strength of an innate immune response to viral infection, which is known to include secretion of human genome-encoded RNases. In such a narrative, patients with weaker immune responses might secrete less defensive RNases, experience less tRNA fragmentation, and develop more severe symptoms.

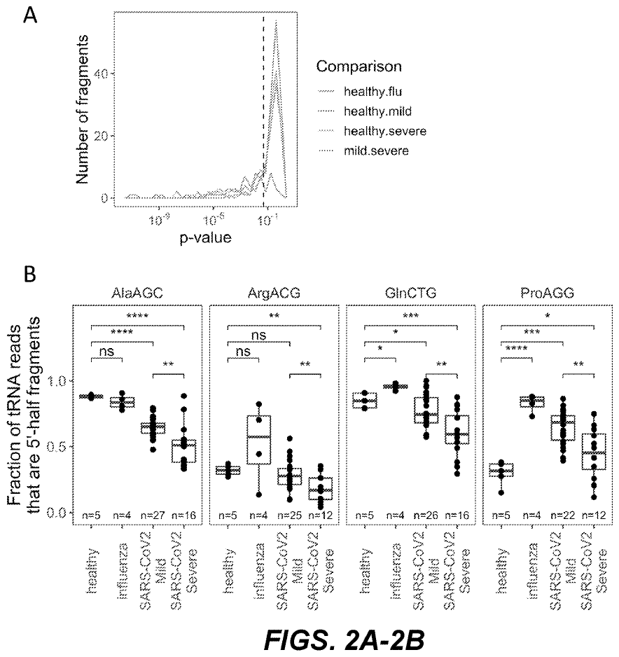

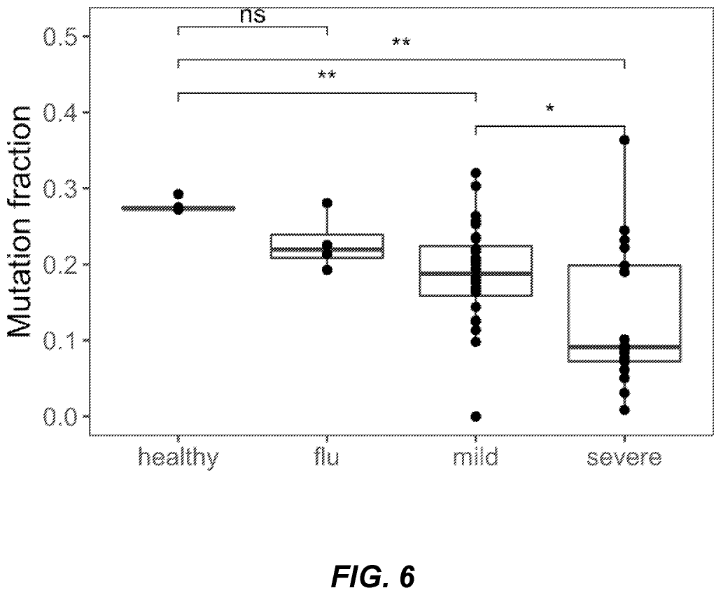

The inventors further analyzed the tRF products of specific tRNAs among all pairwise comparisons of patient groups; samples were filtered for fragments with >10 reads (FIG. 2A). Due to limited cohort sizes, analysis was restricted to the SARS-CoV-2 positive biospecimen. For this analysis, reads from individual isodecoders were pooled among isoacceptor families. This was done because fragment reads often cannot be distinguished among related isodecoder sequences, since the distinguishing bases were cleaved away. Further, RT-qPCR-base assays that can distinguish closely related isodecoders represent an additional challenge, so pooled analysis may yield more transferable insights. Among tRFs of isoacceptor families, statistically significant differences were found among 20 distinct fragments (p<0.05), including fragments for the anticodon loop, variable loop, and T-stem loop. Fragments from the anticodon loop tend to be more abundant and thus better candidates for development into prognostic qPCR tests. Top candidates include tRNA^{Ala}(AGC), tRNA^{Arg}(ACG), tRNA^{Pro}(AGG), tRNA^{Gln}CTG), and tRNA^{Lys}(CTT) (FIGS. 2B, 2C, and 6). For these tRNA isoacceptors, patients who developed mild COVID-19 symptoms produced more tRFs than patients who developed severe COVID-19 symptoms. These results may potentially be associated with the differential activities of immune response in the nasopharyngeal region, for example, the amount of RNase released upon SARS-CoV-2 infection.

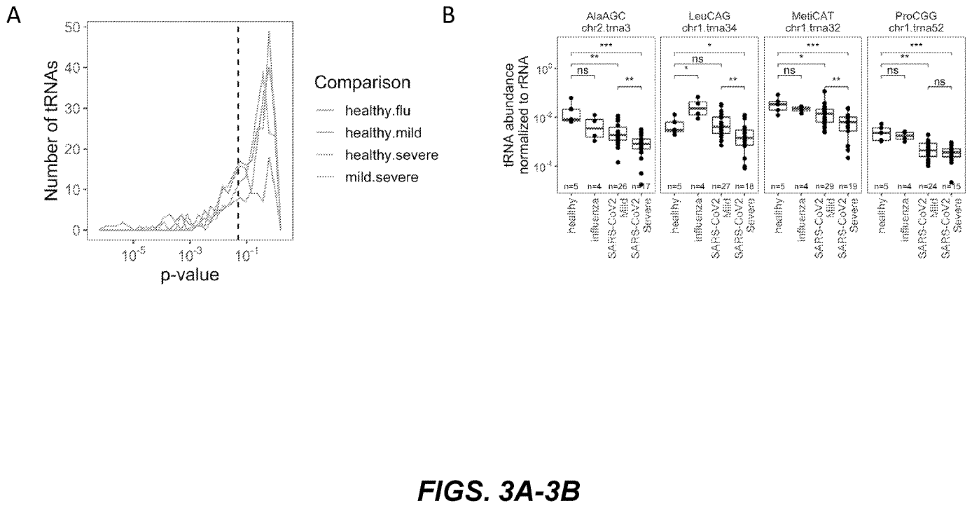

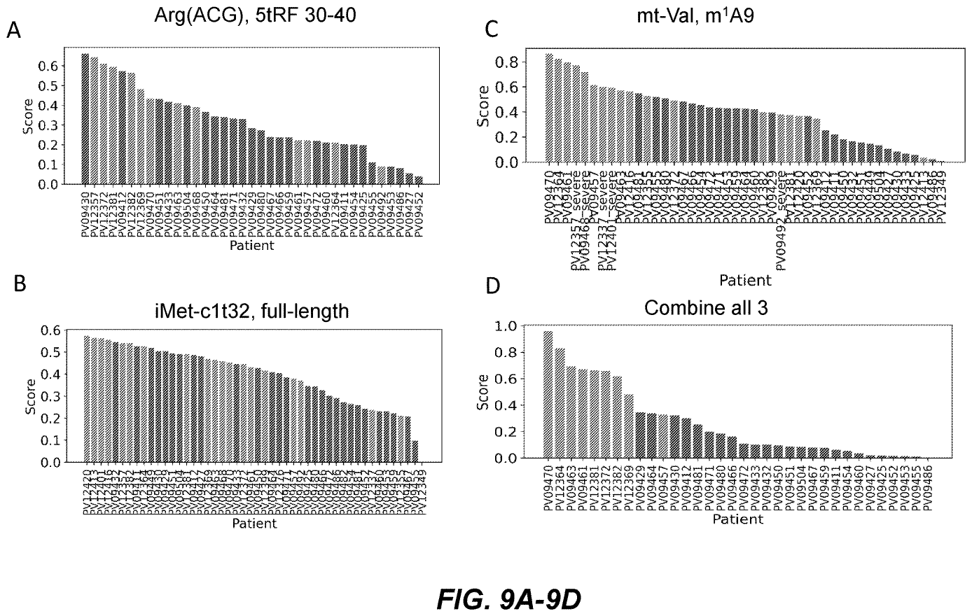

The inventors then analyzed the abundance of full-length tRNAs at the isodecoder level relative to 5.8S rRNA. Abundance was measured relative to 5.8S rRNA, a very abundant small rRNA present in all samples; isodecoders were limited to samples with >10 reads. This choice was made with development of scalable prognostics in mind, where normalization must be done verses a specific, measurable RNA species, as opposed to a “reads per million” approach for typical of RNA-seq analysis. The abundance of all tRNA reads, summed together, compared with 5.8S rRNA was different between uninfected, influenza and SARS-CoV-2 infected groups, though not between SARS-CoV-2 patients (FIG. 9). This may reflect global changes in translation activity within cells or differences in the nature of collected material (e.g. lytic cell debris vs. shed cells). This result also highlights the critical importance of choosing normalization standards. The inventors found significantly different levels of specific tRNA isodecoders in all pairwise comparisons, but the analysis was restricted to the two SARS-CoV-2 groups with the most biospecimen and no global differences in total tRNA abundance. Here, 53 isodecoders showed significant abundance differences between the mild and severe SARS-CoV-2 groups (FIG. 3A). The study highlights three tRNAs that can distinguish between mild and severe SARS-CoV-2 patient groups (FIG. 3B): tRNA^{Ala}(AGC)c2t3, tRNA^{Met}(CAT)c1t32, and tRNA^{Leu}(CGG)c1t34. Further, the study highlights tRNA^{Pro}(CGG)c1t52, which can be used to distinguish SARS-CoV-2 patients from healthy controls. Interestingly tRNA^{Leu}(CGG)c1t34 is not well suited as a general SARS-CoV-2 marker, despite discriminating between symptom groups, demonstrating a diversity of behaviors. Of note, tRNA^{Met}(CAT)c1t32 is the main initiator tRNA isodecoder in human cells (2) and the observed differences in its abundance may reflect the translation activity of the human nasal cells upon SARS-CoV-2 infection. Additionally, tRNAAla(AGC)c6t120 isodecoder allows for discrimination between SARS-CoV-2 infected versus control RNA samples, while tRNAMet(CAT)c1t32 primarily informs the difference between SARS-CoV-2 group I and II. tRNAPro(CGG)c1t52 informs the difference between uninfected control and SARS-CoV-2 infected, as well as group I and II of SARS-CoV-2 infected groups.

tRNAMet(CAT)c1t32, also described herein as MetiCAT, is the main initiator tRNA isodecoder in the human cell (2), and the observed group I and II differences in abundance may reflect the translation activity of the nasal human cells upon SARS-CoV-2 infection.

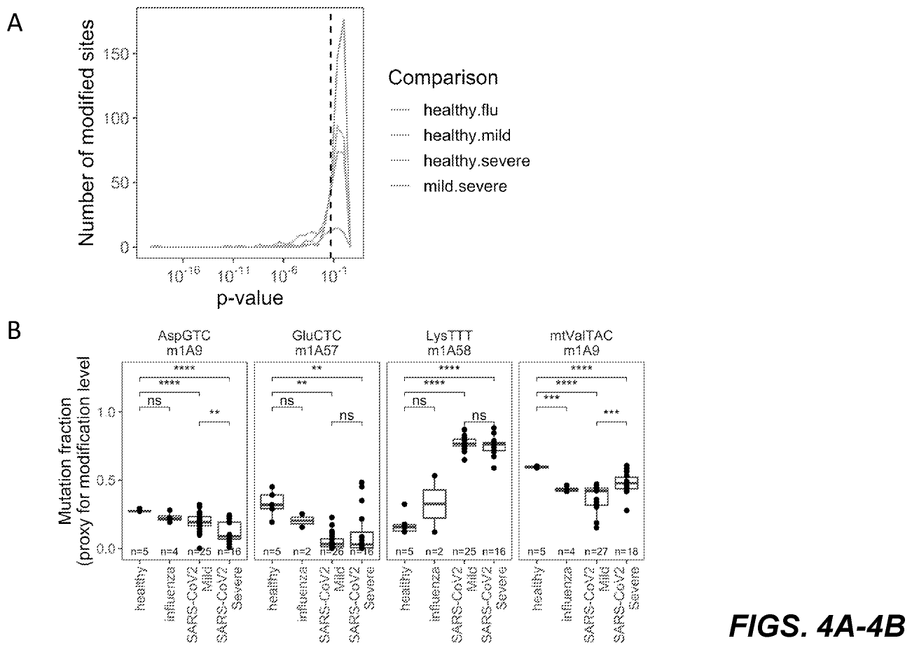

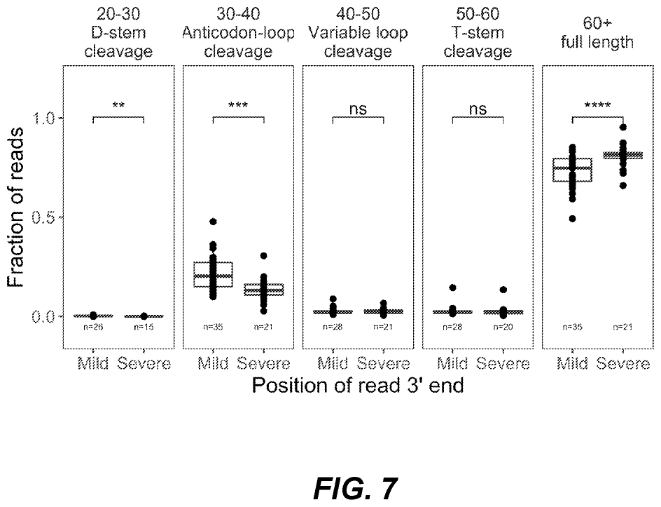

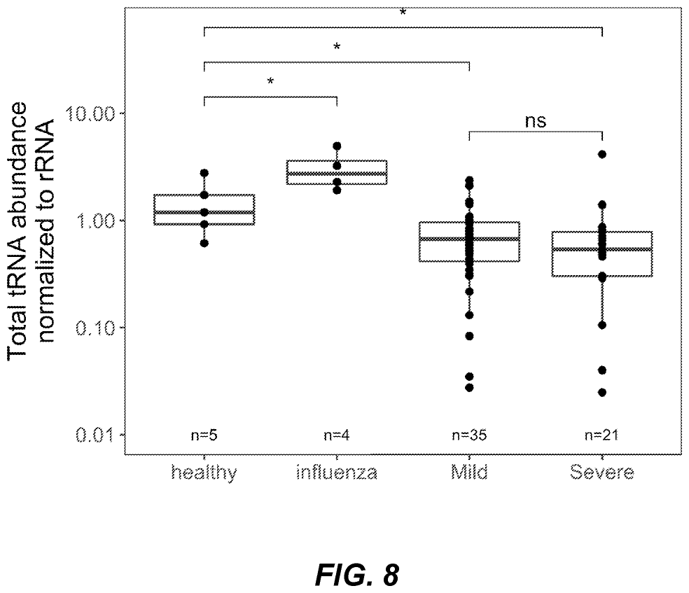

Another parameter obtained from the sequencing data is the comparison of tRNA modification levels through RT mutation signatures (25). Certain modifications interfere with reverse transcription during library preparation, leading to enzymatic misincorporation of bases, which is measured as a “mutation” during sequencing. These signatures do not represent DNA-level mutations, but misincorporation by reverse transcriptase. The mutation rate at specific site can be quantitatively compared to access the differences between modification changes between any two samples. This analysis is particularly sensitive to tRNA modifications with the added chemical group at the Watson-Crick face of the nucleobase, for example, N1-methyladenosine (m¹A, (26, 27)) which is among the most widespread human tRNA modification types. Analysis was limited to sites with a mutation rate >2% and more than 50 reads to limit spurious differences arising from noise at sites known to be unmodified. Again the inventors restrict the analysis to SARS-CoV-2 patients with a greater number of patients sampled. Together, 33 different modification sites showed significant ability to distinguish patient groups, with examples in the D-loop, anticodon loop, and T-stem loop (FIG. 4A). A priori, it is not obvious what effect changes in modification level can have on changes in tRNA biology, including abundance and fragmentation. For example, the inventors previously reported that different modifications can either stimulate or protect from tRNA fragmentation (15). Here the inventors observed a variety of behaviors from different modifications relating to SARS-CoV-2 infection (FIG. 4B). First, m¹A9 on tRNA^{Asp}(GTC) showed progressive reduction of methylation from healthy, to mild symptoms, to severe symptoms patients. Similarly, methylation of m¹A58 on tRNA^{Glu}(CTC) was reduced drastically in all SARS-CoV-2 positive biospecimen compared to those from healthy controls. This site is not suitable to distinguish between SARS-CoV-2 severity groups, likely because the methylation levels are already fully reduced. The opposite behavior was observed for tRNA^{Lys}(TTT) where methylation at m¹A58 dramatically increased upon SARS-CoV-2 infection, but again, methylation cannot distinguish among SARS-CoV-2 patients. Finally, methylation of m¹A9 of mt-tRNA^{Val}(TAC) followed a pattern remittent of fragmentation: methylation was significantly reduced in samples obtained from healthy controls compared to samples from patients with mild COVID-19. This response was further muted in biospecimen from patients with severe COVID-19. Of note, mitochondrial tRNA^{Val} plays a dual role in mitochondrial translation. It not only works as a tRNA in translation but is also an essential component of the human mitochondrial ribosome (28). M¹A9 in mt-tRNA^{Val} may, thus, modulate its activity either as a tRNA and/or its ribosomal function. This could plausibly be related to global changes in translation reflected in FIG. 6. The m¹A9 mutation fraction of a mitochondrial tRNA is particularly useful in separating SARS-CoV-2 group I and II. m¹A9 is also present in the cytosolic tRNAAsp which showed a significant difference between the two groups of SARS-CoV-2 infected people (FIG. 7).

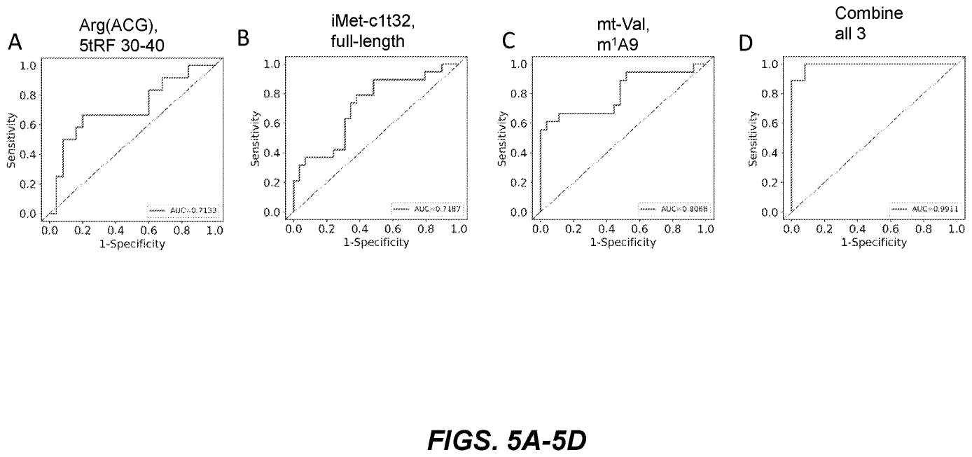

Based on the findings described above, it was hypothesized that these tRNA features could be used as biomarkers. To test this hypothesis, the inventors selected specific tRNAs for fragmentation, abundance, and modification for statistical calculation of COVID-19 severity (FIGS. 5A-5D). Each of the three individual parameters produced an area under curve (AUC) value of 0.71-0.84. Combining all three tRNA parameters produced an AUC value of 0.99 (FIG. 5D), indicating the power of using multiple tRNA properties for accurate prediction of SACS-CoV-2 infection symposium severity.

In summary, the inventors were able to generate good quality tRNA-seq results from residual diagnostic nasopharyngeal swab biospecimens. tRNA profiles of these RNA samples, taken at the time of initial SARS-CoV-2 diagnosis, may provide new information that can be used to predict COVID-19 symptom severity. These results represent a distinct approach in defining biomarkers of infectious disease severity which may allow for the identification of patients at high risk for complication from respiratory virus infection.

### Example 2: Template Ligation qPCR (TL-qPCR) for Detection of m1A Modifications

N1-methyladenosine (m¹A) is among the most abundant eukaryotic RNA modification present in tRNA, rRNA, mRNA. In humans, m¹A is present in all cytosolic tRNA and 15/22 mitochondrial tRNAs. M1A plays a role in tRNA stability and in translational fine-tuning. M1A modification levels in specific tRNAs can also be used as biomarkers for clinical prognosis such as development of COVID-19 severity. Thus, the ability to quantify m¹A modification rapidly and quantitatively in individual tRNA will be a valuable tool for diagnostic applications.

M1A modifications in RNA are measured either by mass spectrometry (MS), thin-layer chromatography (TLC), or reverse transcription (RT). LC-MS and TLC methods generally measures the total m1A levels in bulk RNA; individual tRNA m1A level can only be measured after its isolation from bulk RNA which is laborious, time consuming, and requires large amount of input material. RT method measures m1A in individual tRNA through m1A-induced stop which can be visualized and quantified by gel electrophoresis. RT method can also be used in high throughput sequencing by quantifying the “mutated” reads at the m1A site induced by the read-through of m1A of the reverse transcriptase. The mutational profiling with sequencing (MaP-seq) has been widely used to study m1A modifications in RNA. Although powerful, sequencing of tRNA is still slow to implement, expensive, and requires at least tens of nanograms of the input RNA.

Here the inventors describe a template ligation and quantitative PCR (TL-qPCR) method to quantify m¹A modification at single base resolution. TL-qPCR does not involve reverse transcription, rather, it detects and quantifies tRNA and its m¹A modification through splint ligation using the very efficient SplintR ligase using tRNA as the template. The ligated DNA product is then amplified and quantified by real-time PCR. The avoidance of using reverse transcriptase in the method bypasses a major cause of inconsistencies in quantitative tRNA analysis. The inventors show that SplintR ligation works well for tRNA with very low sample input. More importantly, the inventors develop the method for the new application to quantify the levels of m¹A modification in specific tRNA in cellular total RNA samples which can be useful for biomarker developments using clinical samples.

tRNA is the most abundant RNA species in a total RNA sample by molarity. tRNA is also extensively modified with over 100 various modifications. Current methods of detecting tRNAs and tRNA modifications include Northern blot (Khalique et al., 2022), next-generation sequencing (Clark et al., 2016; Zheng et al., 2015), and end-ligation based qPCR (Honda et al., 2015) etc. A SplintR ligation-based qPCR method has been developed to detect microRNA (miRNA) (Jin et al., 2016; Krzywkowski and Nilsson, 2017). This method relies on the template splint ligation by SplintR ligase, which miRNA serves as the template. The ligation efficiency of template splint ligation by SplintR is very high comparing to previously established methods of using T4 RNA ligase 2 or T4 DNA ligase. In addition, the ligation efficiency of SplintR is negligible without the template. Thus, the ligation efficiency is proportional to the abundance of the miRNA.

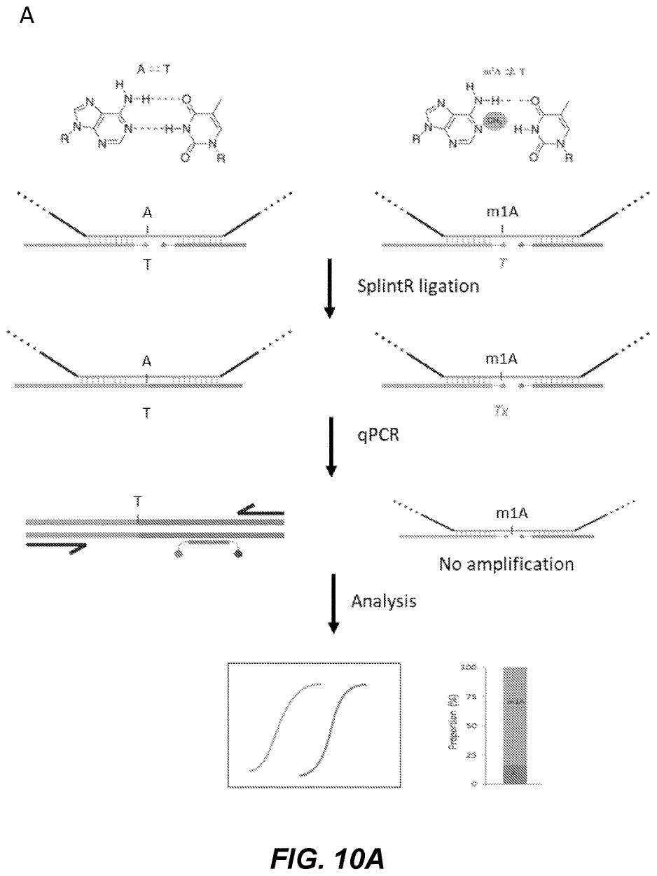

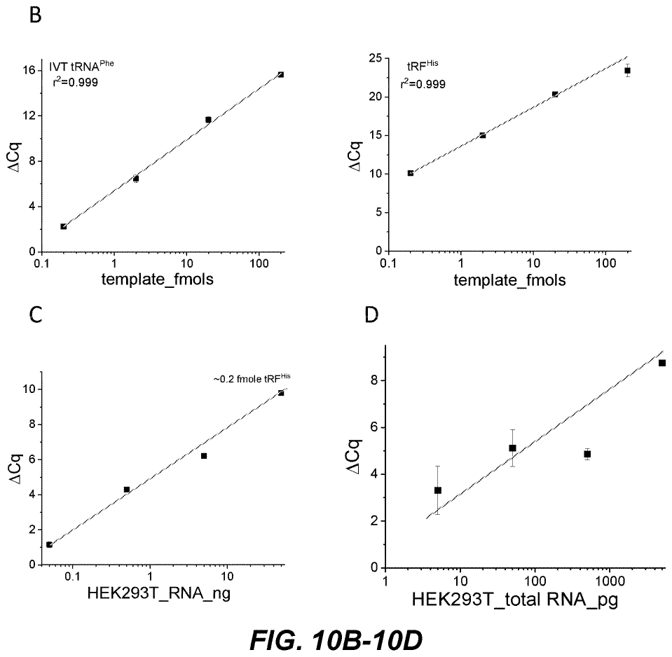

The inventors took advantage of the SplintR ligation selectivity and developed a template ligation qPCR assay for the detection of 1-methyl adenosine (m¹A) modification in tRNAs (FIG. 10A). The inventors reasoned that m¹A modification has a methyl group at the N1 position of the Watson-Crick face of Adenosine (A), which affects the base pairing between A and T. This may block the template splint ligation by SplintR. The inventors designed two pairs of linker oligos that bind to the tRNA template near or far away from the m¹A modification site. For the linker oligos near the modification site, the 5′ end of the upstream linker (5′ linker, 5lkr) has a phosphorylated T that can binds to the complementary template containing A or m¹A. The downstream linker (3′ linker, 3lkr) has a fluorescent probe binding site and a PCR primer binding site. The other pair of linker oligos bind to the complementary template at the position without known modifications nearby and serve as the endogenous control. After SplintR ligation, the amount of ligated product can then be detected by qPCR with fluorescent probes. The m¹A modification fraction can be calculated based on the qPCR threshold (Cq) values of the two SplintR ligation samples.

To test the sensitivity of TL-qPCR, the inventors used the in vitro transcript of yeast tRNA^{Phe}, a synthetic RNA oligonucleotide corresponding to the human 5′ tRNA^{His} fragment, and tRNA^{iMet} in total human RNA. The ΔCq of the two unmodified RNAs was proportional to the input amount of 0.2-200 fmole (FIG. 10B). Using the human tRF^{His} set up, the inventors detected and quantified the amount of tRF^{His} in HEK293T total RNA (FIG. 10C) which is estimated to be present at 0.1-0.5 fmole in 50 ng total RNA (ref.). The inventors also applied TL-qPCR for measuring tRNA^{iMet} in total human RNA from a cell line (FIG. 10D) and detected tRNA^{iMet} in as little as 5 pg HEK293T total RNA input. These results validate the principle of the TL-qPCR design for unmodified tRNA, small RNA, and native tRNA.

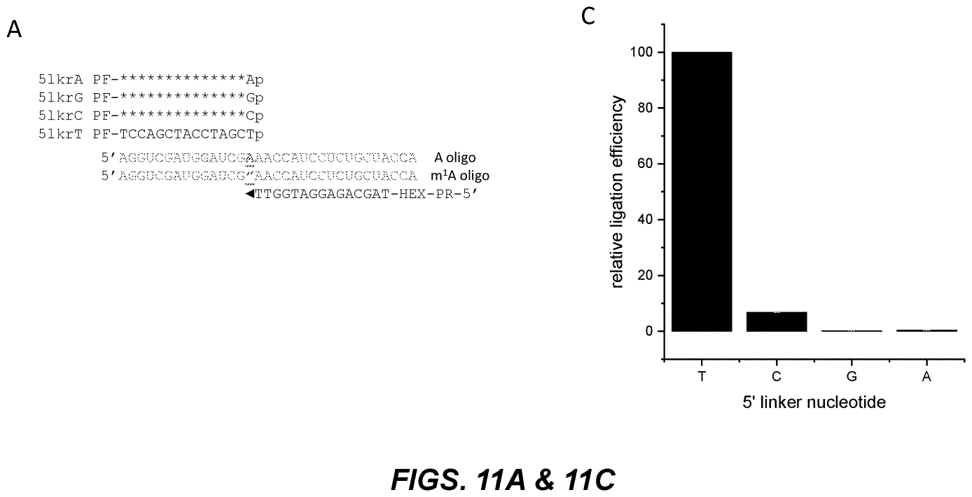

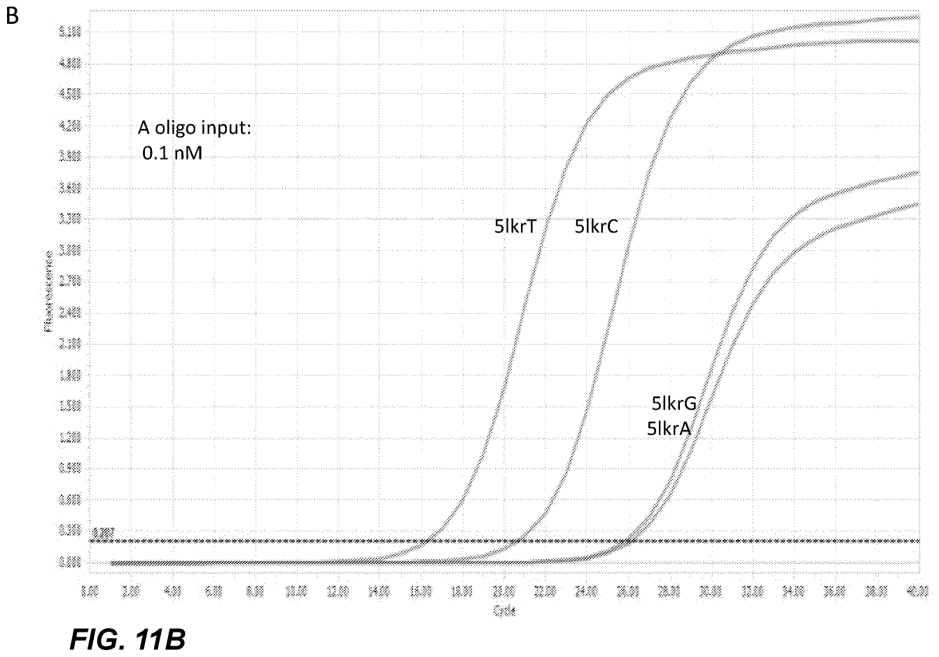

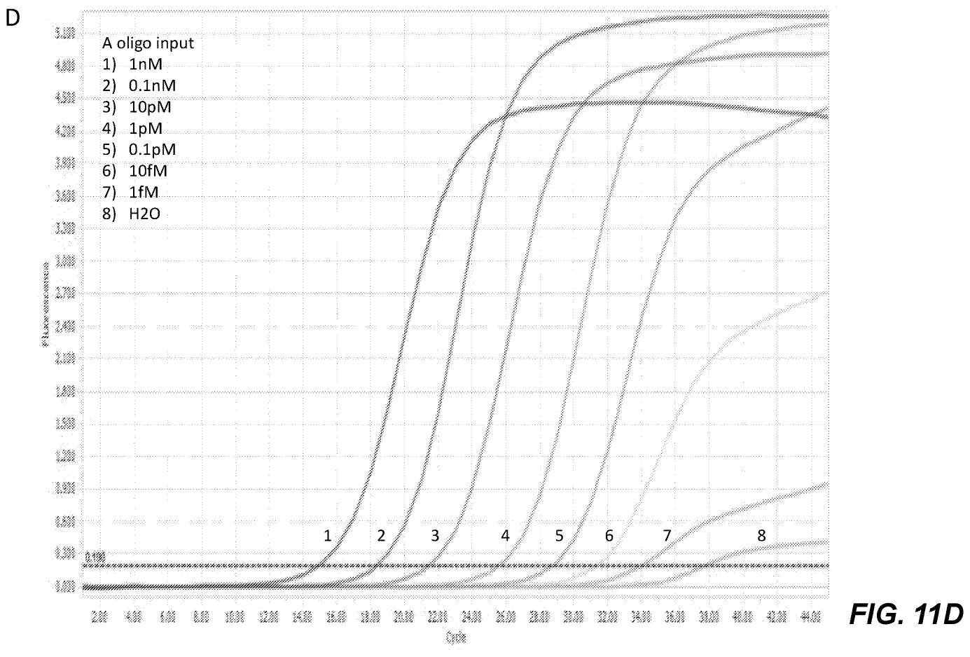

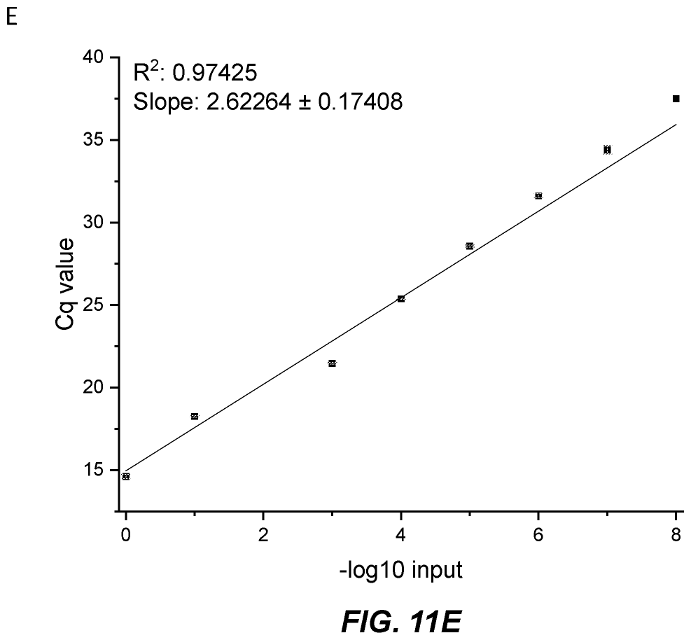

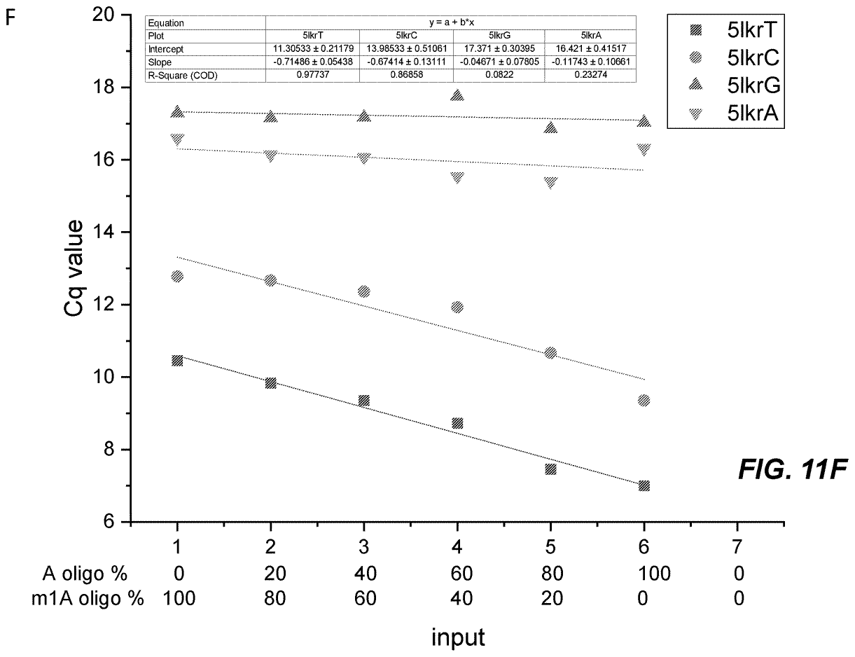

To demonstrate the feasibility of the TL-qPCR method, the inventors applied the method to study the m¹A modification in synthetic oligos that have identical sequences to the m¹A58 surrounding region of human tRNA^{iMet}. First, the inventors tested whether one nucleotide mismatch at the ligation site affect the template splint ligation efficiency using unmodified RNA oligo. The inventors designed four different 5′ linker oligos with T, C, G or A at the 5′end (FIG. 11A). The TL-qPCR profile showed that one nucleotide mismatch was sufficient to suppress the template splint ligation by SplintR (FIG. 11B). The relative amounts of PCR product for C, G and A-ending 5′ linker oligos were less than 10% compared to the T-ending 5′ linker, (FIG. 11C). This result demonstrates that one nucleotide mismatch can reduce the template splint ligation significantly. The results are crucial for tuning the TL-qPCR strategy since the method is based on the impaired base pairing between m¹A and T at single nucleotide. Next, the inventors tested whether this method can quantify the abundance of the template ligated product by varying the input amount of the RNA oligo. The qPCR amplification profile of serial dilution of the unmodified oligo showed that the Cq values increased proportionally as the input amount was reduced (FIG. 11D). The standard curve of the serial diluted samples showed an excellent linear correlation between the Cq value and the RNA oligo input (FIG. 11E). This result demonstrates that the method can quantitatively measure the template splint ligated product.

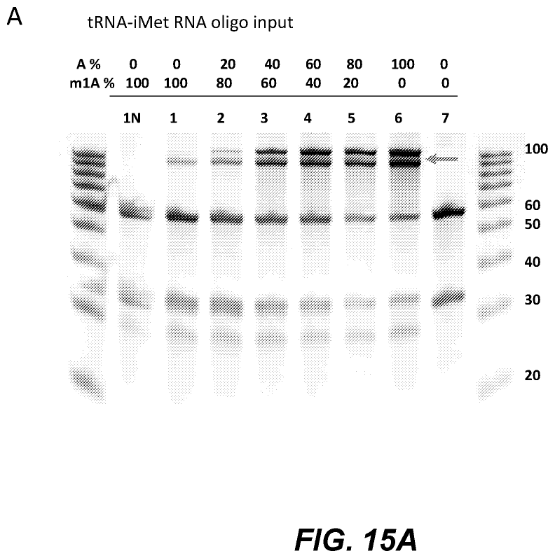

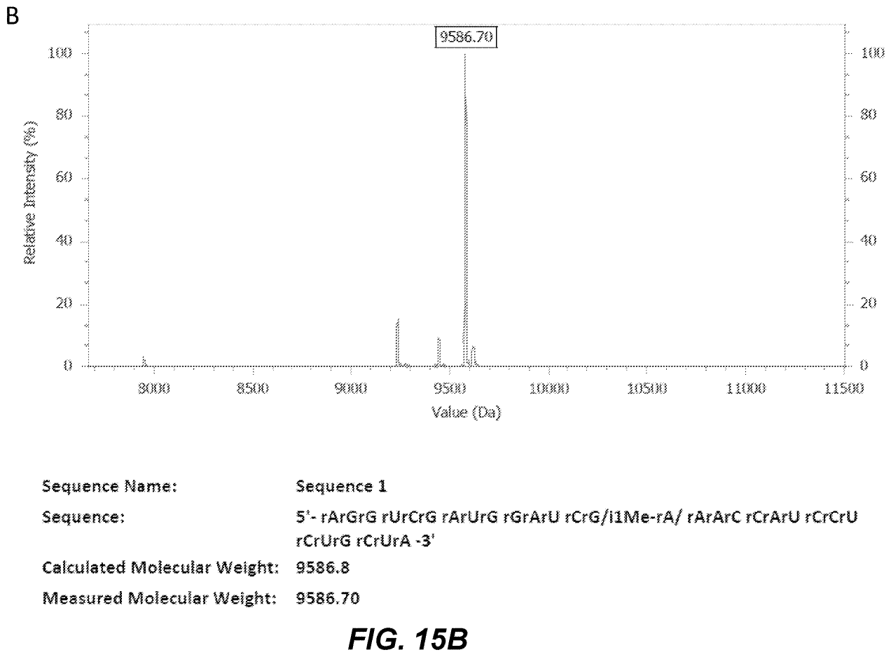

To investigate the quantitative nature of TL-qPCR for m1A levels, the inventors varied the ratio of a synthetic m¹A modified oligo and unmodified oligo in the input and performed TL-qPCR with 5′ linker oligos that have T, C, G or A at the 5′end. Cq values obtained using T-ending 5′ linker oligo showed the best linear correlation (R²=0.977) with the ratio of m¹A RNA oligo compared to other 5′ linker oligos (FIG. 11F). However, the sample with supposedly 100% m¹A oligo still showed low amount of TL-qPCR product, and a low level of ligation product was detectable using this oligo (FIG. 15A). This result may be derived either from incomplete suppression of ligation by m¹A modification, the residual presence of unmodified oligo, or a known m¹A-to-m⁶A migration during oligo synthesis. ESI-MS showed that the commercial m¹A oligo did not contain unmodified oligo but was consistent with the presence of m⁶A (FIG. 15B). Nevertheless, these results demonstrate that the TL-qPCR method can quantify m¹A modification fraction in model oligos.

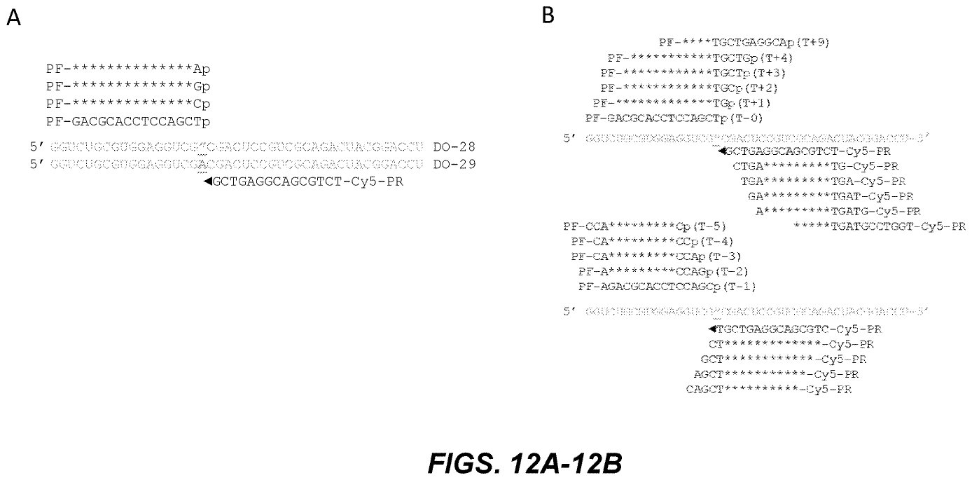

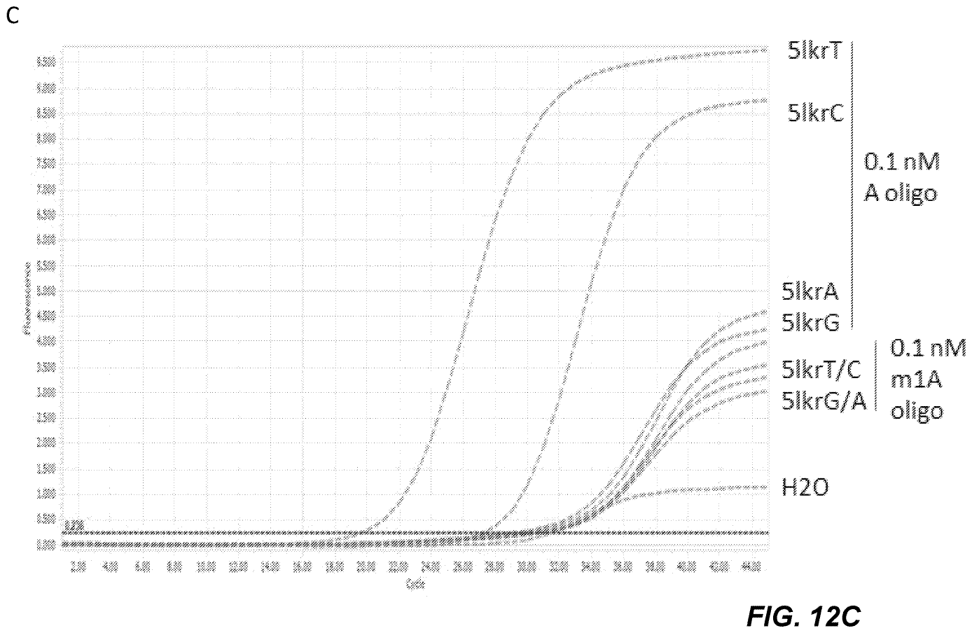

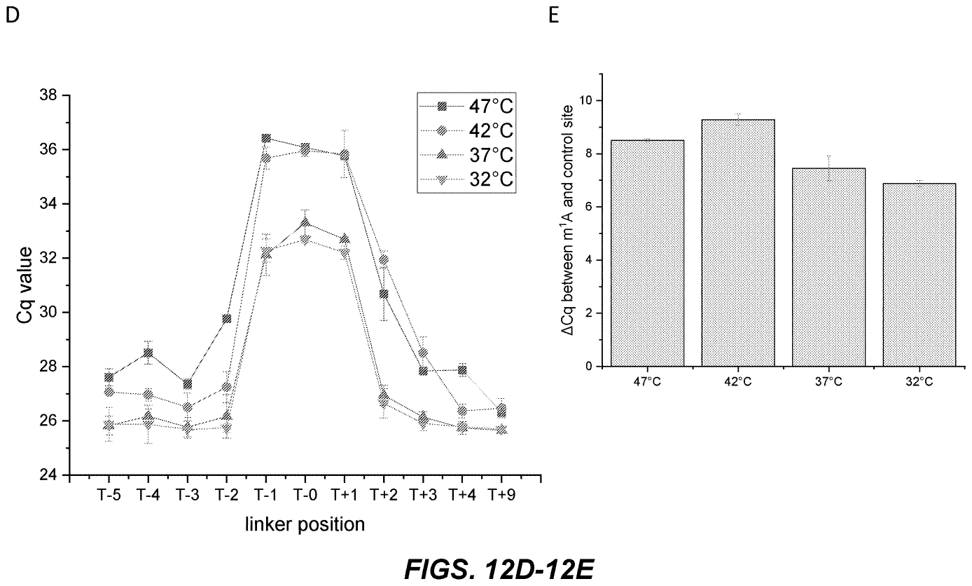

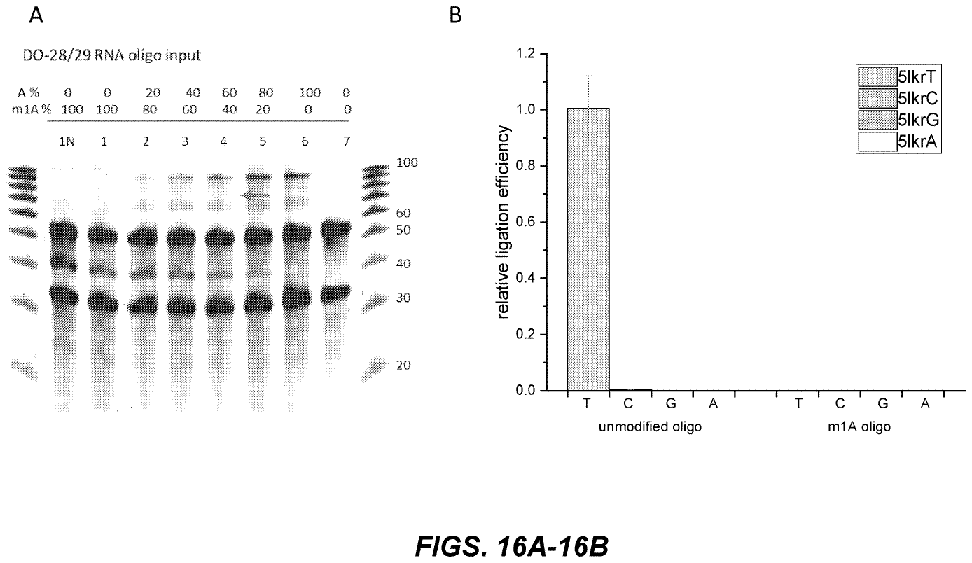

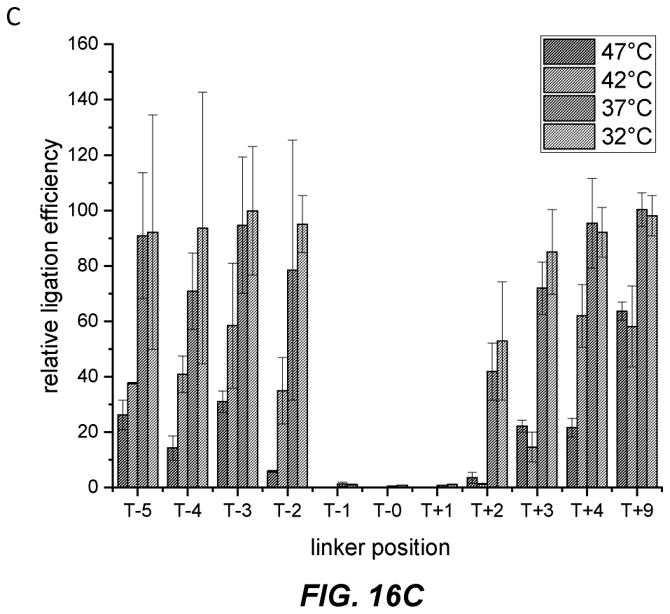

The inventors further investigated the context dependence of the m¹A modification effects using another pair of unmodified and m¹A modified oligos (D028A and DO29m¹A) there were synthesized using an improved method that minimized m¹A-to-m⁶A conversion. The inventors found no ligated product using only the m¹A-modified oligo (FIG. 16A), indicating that m¹A can nearly fully block templated splint ligation. Furthermore, the ligated product was proportional to the ratio of unmodified oligo in total RNA mixture. The inventors then performed TL-qPCR with 5′ linker oligos with different 5′ end using m¹A modified or unmodified oligos (FIG. 12A). The Cq values obtained with m¹A modified oligo and all 5′ linker oligos were lower than the Cq oligos obtained with unmodified oligos (FIG. 12B, FIG. 16B), consistent with the result above using the tRNAᵢ^{Met}-mimicking oligo.

To quantify m¹A modification, the inventors also need to include a template splint ligation control in the same RNA that is not affected by the m¹A modification to measure the overall abundance of both modified and unmodified RNA. The control ligation site should be close to the m¹A ligation site so that the inventors can use the same primers and probes for qPCR, thus reducing the bias caused by PCR primers and probes. The inventors shifted the ligation site by N nucleotides upstream (T−N) or downstream (T+N) of the target m¹A ligation site and design the corresponding linker oligos (FIG. 12C). Using these oligos the inventors also tested how temperature of the splint ligation reaction from 32-47° C. affected the specificity and sensitivity of the method. Temperature optimization is important for quantifying m¹A modification in RNA where changing the length of the linker oligos in the template splint ligation reaction at higher temperatures increases specificity of targeting specific RNA. As expected, m¹A modification affected TL-qPCR when using ligation oligos within one nucleotide of m¹A. When the ligation site was 3-5 nucleotides away either upstream or downstream from the m¹A ligation site the Cq value differences reached a plateau (FIG. 12D). Increasing the ligation reaction temperature decreased the TL-qPCR product amounts but still maintained the specificity according to the Cq value difference between the distal ligation site and target m¹A ligation site (FIG. 12D, FIG. 12E, FIG. 16C). Based on these results, the inventors chose 37° C. for template splint ligation and the control site ≥4 nucleotide away from the m¹A site for the following studies.

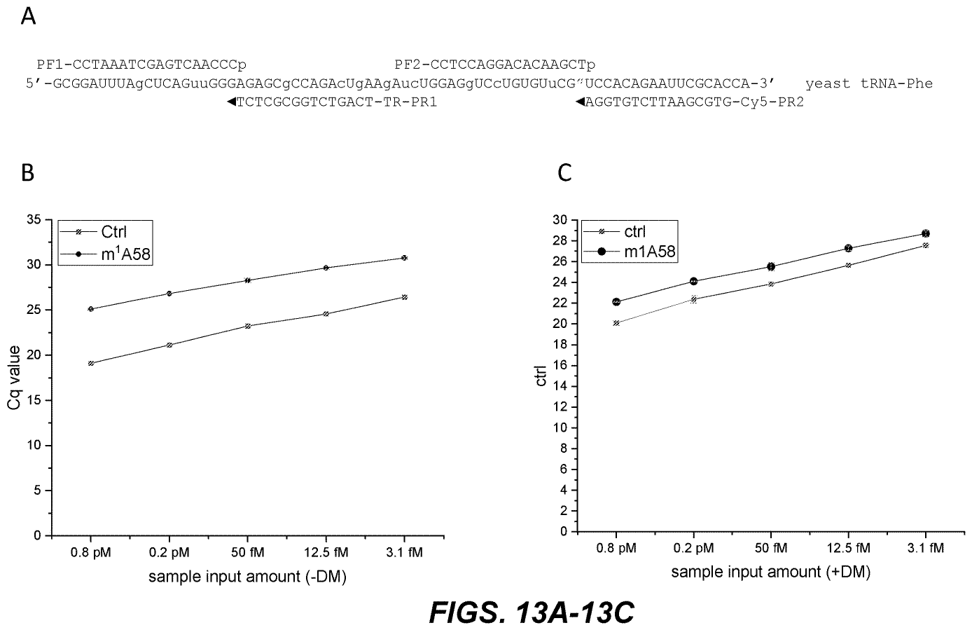

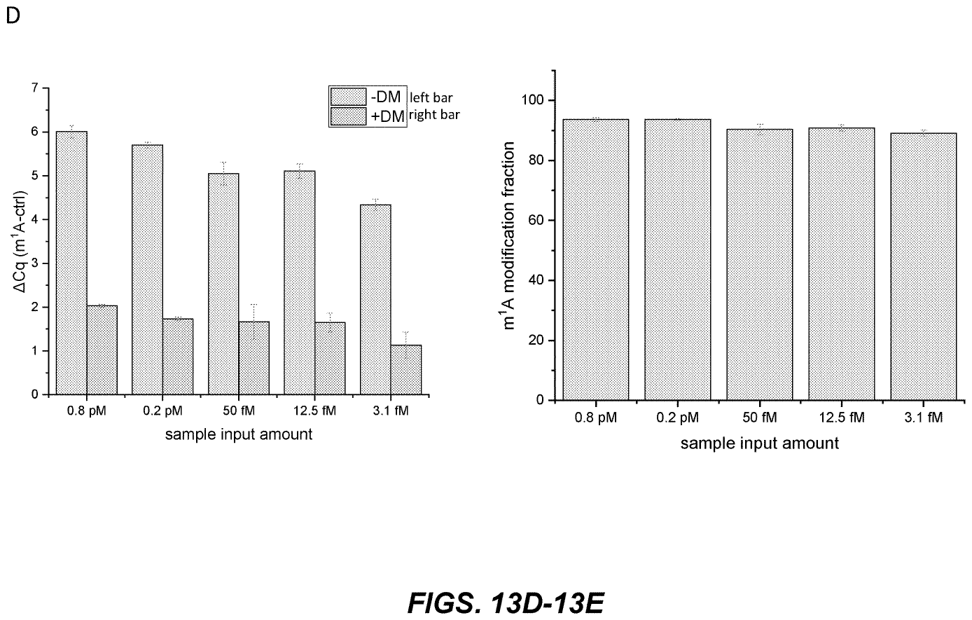

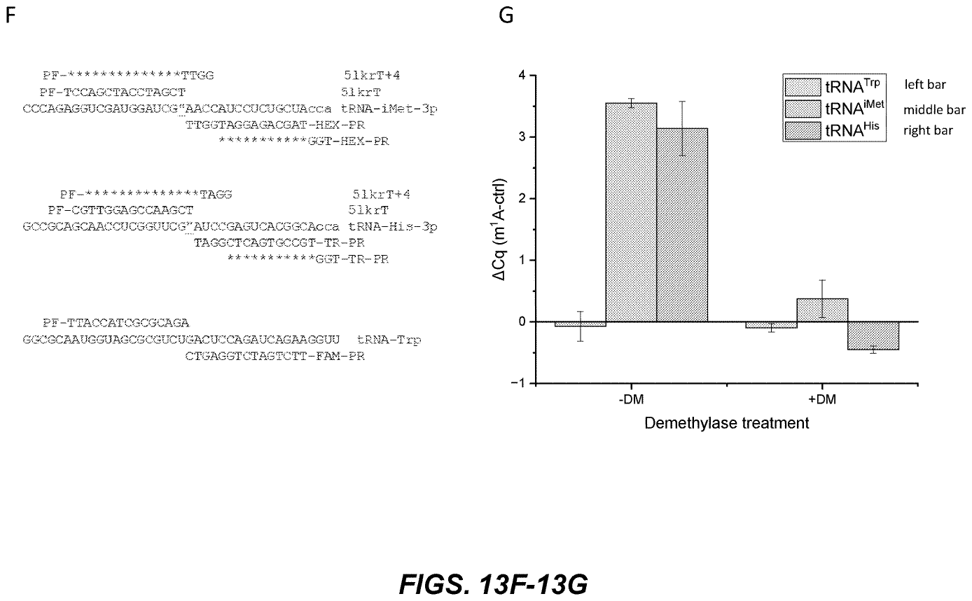

The inventors applied the method next to detect and quantify m¹A58 modification in yeast tRNA^{Phe}. The inventors designed the linker oligos at the m¹A site and at another site away from the modification site (control) with different fluorescent probe binding site for multiplex qPCR (FIG. 13A). The inventors performed TL-qPCR on 4× serial diluted yeast tRNA^{Phe} samples. The Cq values of both the control and m¹A ligation sites increased as the input RNA amount decreased (FIG. 13B). To validate that the m¹A58 targeting oligos were indeed responsive to m¹A, the inventors partially removed the m¹A modification using the E. coli AlkB demethylase and used the demethylase-treated samples as templates for TL-qPCR. The Cq values of control sites did not change much, but the Cq values of the m¹A58 sites were significantly reduced, confirming that the m1A58 targeting oligos indeed reports the m¹A58 modification in yeast tRNA^{Phe} (FIG. 13C). Furthermore, the differential Cq value (ΔCq) between m¹A site and control site for the demethylase-treated (i.e. less m1A modification) and untreated (ΔΔCq) remained constant when varying the input RNA from 800 to 3.1 fmole (2.2 ng to 0.08 ng), indicating the robustness of the method when applied to a natural tRNA at the level as low as 3.1 fmole tRNA (FIG. 13D).

The inventors used the ΔΔCq values to calculate the m¹A modification fraction as follows.

$${m}^{1} A \% = 100 \% - A \%$$

$$A \% = 100 \% \times {2}^{- ΔΔ Cq}$$

$$ΔΔ Cq = Δ {Cq}_{- DM} - Δ {Cq}_{+ DM}$$

$$Δ Cq = {Cq}_{{m}^{1} A} - {Cq}_{ctrl}$$

The inventors obtained yeast tRNA^{Phe} m¹A58 modification fraction at 90-93% level (FIG. 13E) which is close to that measured by primer extension using AMV RT stop of ~97%.

The inventors next tested the method for the m¹A58 modifications in human tRNAs using total RNA as input. Because the yeast tRNA^{Phe} sample was from partially purified from total RNA, this experiment tested the ability to directly measure m¹A58 in a complex mixture of biological samples. The inventors chose three target tRNAs for the linker oligo design with different fluorescent probe binding region for m¹A sites and control sites (FIG. 13F) which enabled three color-qPCR to measure 3 tRNAs simultaneously. For m¹A sites the inventors chose to study human tRNA^{iMet} and tRNA^{His} which are of biological interest in previous studies (ref.), for example, tRNA^{iMet} m¹A is a biomarker for COVID symptom severity (ref.). In addition, the inventors also designed the linker oligos for tRNA^{Trp} at a region with no known m¹A modifications which used tRNA^{Trp} in the same sample as an external control. The inventors performed TL-qPCR using total RNA as template and with and without demethylase treatment. For the control tRNA^{Trp} site, demethylase treatment did not change the ΔCq value confirming the unmodified status of the ligation site. However, the ΔCq values of the tRNA^{iMet} and tRNA^{His} m¹A sites were significantly reduced after demethylase treatment, demonstrating the presence of m¹A modifications (FIG. 13G).

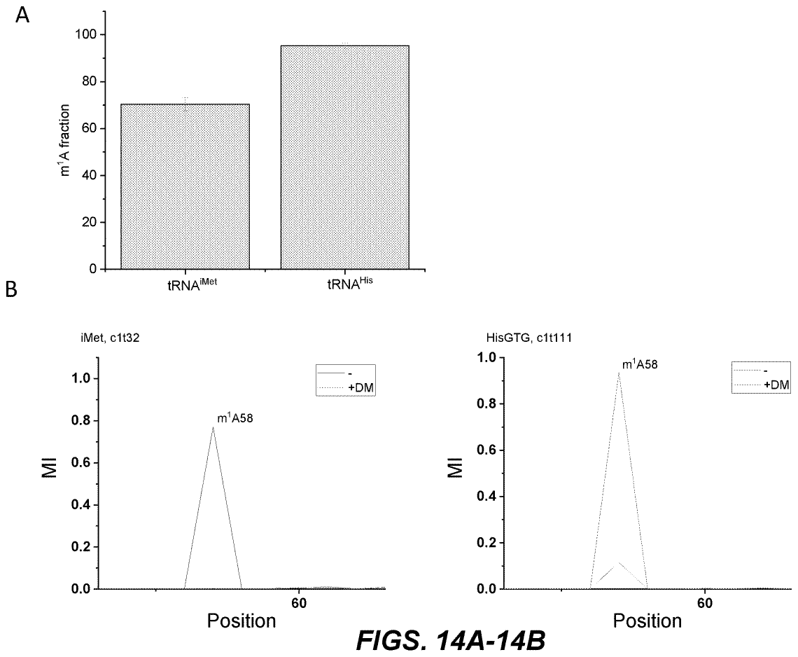

To examine the approach to measure m1A modification fraction of individual tRNA in biological RNA, the inventors collected fresh total RNA samples from HEK293T cells and performed TL-qPCR to quantify the m¹A58 modification fraction of tRNA^{iMet} and tRNA^{His}. The inventors used three-color scheme in FIG. 4F that simultaneously measured the m¹A58 modification status of tRNA^{iMet} and tRNA^{His} and the tRNA^{Trp} control. The inventors obtained the m¹A58 modification fraction for tRNA^{iMet} and tRNA^{His} as 70.3±2.8% and 95.3±1.2%, respectively (FIG. 14A). The inventors have shown previously that a reverse transcription and sequencing based method by DM-tRNA-seq could quantitatively report m¹A58 modification fractions in HEK293T cells (ref.). In that sequencing study, the m¹A58 modification fractions for tRNA^{iMet} and tRNA^{His} were estimated to be 76% and 95%, respectively (FIG. 14B). The excellent correlation between TL-qPCR and RT-sequencing demonstrates the effectiveness and accuracy of the TL-qPCR method in detecting and quantifying m¹A modification fraction in tRNA.

In summary, the inventors developed a template splint ligation-based qPCR method for the detection and quantification of m¹A modifications in tRNA. Certain methods described herein rely on the specificity and sensitivity of the SplintR ligase. A single base mismatch or inefficient binding of the ligation oligos caused by Watson-Crick face modifications is sufficient to vastly reduce or block the template splint ligation by SplintR ligase. Furthermore, other RNA modifications such as pseudouridine (Ψ) may also be studied using TL-qPCR after chemical treatments that interfere with Watson-Crick base pairing. The method can quantitatively access m¹A modification in as low as 3 fmol tRNA or ~80 pg. In addition to RNA modification detection, the method can also be used to detect the abundance of tRNAs or other small RNA biomarkers in disease models. The method can be performed in a matter of hours, easy to operate, and requires less hand-on time compared to sequencing or primer extension followed by gel electrophoresis. The ease of using one-day protocol for target RNA modification or abundance detection should enable widespread application of the TL-qPCR method for tRNA and tRNA modification studies.

### Materials and Methods for TL-qPCR for Various Aspects Described Herein

### Oligonucleotide Design

The RNA and DNA oligonucleotides used in the study were purchased from IDT (Integrated DNA Technologies, Inc., Coralville, IA, USA) using standard desalting purification. The m¹A or A containing oligos (DO-28/29) were synthesized in the lab using standard DNA oligo synthesis methods.

Two sets of SplintR ligation linkers (5′ linkers and 3′ linkers) were designed for each m¹A site with one set of linker oligos targeting the m¹A site and the other set targeting an upstream or downstream control region. The 5′ linker oligos were phosphorylated at the 5′end to enable ligation. A primer-binding sequence for forward or reverse primer during PCR, and a synthetic Taqman probe binding sequence was designed as just at the downstream of reverse primer-binding site in 3′ linker oligo.

Synthetic primers and probes were used in qPCR for m¹A quantification, which were all designed as artificial sequences without complementary matching to any human genomic DNA or RNA sequences. A set of primers and a fluorescence-labeled probe were designed for each m¹A site to enable multiplex qPCR (Cy5, Texas Red and FAM).

### HEK293 Cell Total RNA and Demethylase Treatment

HEK293T cells were cultured in DMEM medium (Cytiva, SH30022.01) with 10% FBS and 1% Pen-Strep (Penicillin-Streptomycin) to 80% confluency (Zhang et al., 2019). Total RNA was extracted using TRIzol reagent according to the manufacturer's manual. Demethylase treatment was performed as reported before (Watkins et al., 2022).

### SplintR Ligation

Template SplintR ligation was performed as follows. 2 μl of input RNA oligos the (final concentration was from 1 nM to 0.1 pM by 10× serial dilution) and 2 μl of linker oligos mix 100 nM each linker was incubated in a heated-lid thermocycler at 90° C. for 2 min. For HEK293T total RNA samples, the final concentration ranged from 40 ng/μl to 2 ng/μl. After the denaturation, the mix was decreased to 40° C. in 5 mins gradually. Then the ligation mix containing 5 U of PBCV-1 DNA ligase (SplintR, M0375S, NEB) and 1× SplintR buffer was added to the annealed primer/RNA mix to a final volume of 10 μl. The ligation mixture was then incubated at 37° C. for 30 min. For temperature dependent template splint ligation by SplintR, the samples were incubated at 32° C., 37° C., 42° C., or 47° C. for 30 mins, respectively. After ligation the mix was then incubated at 68° C. for 5 mins to denature the ligase.

qPCR

All qPCR experiments were performed on LightCycler Real-time PCR system, using 2×qPCR Master Mix Master mix (IDT, 1055772) with specific primers and probes. The thermocycling conditions were as follows: Initial denaturation at 95° C. for 2 min, followed by 42 cycles of amplification at 95° C. for 10 sec and 57° C. for 30 sec. Data were analyzed using the ΔΔCq method after normalized to endogenous control.

All of the methods disclosed and claimed herein can be made and executed without undue experimentation in light of the present disclosure. While the compositions and methods of this invention have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Boccaletto, P., Stefaniak, F., Ray, A., Cappannini, A., Mukherjee, S., Purta, E., Kurkowska, M., Shirvanizadeh, N., Destefanis, E., Groza, P. et al. (2022) MODOMICS: a database of RNA modification pathways. 2021 update. Nucleic Acids Res, 50, D231-D235.
2. Chan, P. P. and Lowe, T. M. (2016) GtRNAdb 2.0: an expanded database of transfer RNA genes identified in complete and draft genomes. Nucleic Acids Res, 44, D184-189.
3. Gu, C., Begley, T. J. and Dedon, P. C. (2014) tRNA modifications regulate translation during cellular stress. FEBS Lett, 588, 4287-4296.
4. Begley, U., Dyavaiah, M., Patil, A., Rooney, J. P., DiRenzo, D., Young, C. M., Conklin, D. S., Zitomer, R. S. and Begley, T. J. (2007) Trm9-catalyzed tRNA modifications link translation to the DNA damage response. Mol Cell, 28, 860-870.
5. Zhang, W., Foo, M., Eren, A. M. and Pan, T. (2022) tRNA modification dynamics from individual organisms to metaepitranscriptomics of microbiomes. Mol Cell, 82, 891-906.
6. Huang, H. Y. and Hopper, A. K. (2016) Multiple Layers of Stress-Induced Regulation in tRNA Biology. Life (Basel), 6, 16.
7. Oberbauer, V. and Schaefer, M. R. (2018) tRNA-Derived Small RNAs: Biogenesis, Modification, Function and Potential Impact on Human Disease Development. Genes (Basel), 9, 607.
8. Anderson, P. and Ivanov, P. (2014) tRNA fragments in human health and disease. FEBS Lett, 588, 4297-4304.
9. Schimmel, P. (2018) The emerging complexity of the tRNA world: mammalian tRNAs beyond protein synthesis. Nat Rev Mol Cell Biol, 19, 45-58.
10. Pandey, K. K., Madhry, D., Ravi Kumar, Y. S., Malvankar, S., Sapra, L., Srivastava, R. K., Bhattacharyya, S. and Verma, B. (2021) Regulatory roles of tRNA-derived RNA fragments in human pathophysiology. Mol Ther Nucleic Acids, 26, 161-173.
11. Nunes, A., Ribeiro, D. R., Marques, M., Santos, M. A. S., Ribeiro, D. and Soares, A. R. (2020) Emerging Roles of tRNAs in RNA Virus Infections. Trends Biochem Sci, 45, 794-805.
12. Jin, D. and Musier-Forsyth, K. (2019) Role of host tRNAs and aminoacyl-tRNA synthetases in retroviral replication. J Biol Chem, 294, 5352-5364.
13. Koczera, P., Martin, L., Marx, G. and Schuerholz, T. (2016) The Ribonuclease A Superfamily in Humans: Canonical RNases as the Buttress of Innate Immunity. Int J Mol Sci, 17, 1278.
14. Lu, L., Li, J., Moussaoui, M. and Boix, E. (2018) Immune Modulation by Human Secreted RNases at the Extracellular Space. Front Immunol, 9, 1012.
15. Watkins, C. P., Zhang, W., Wylder, A. C., Katanski, C. D. and Pan, T. (2022) A multiplex platform for small RNA sequencing elucidates multifaceted tRNA stress response and translational regulation. Nature Communications, 13, 2491.
16. Javaid, W., Ehni, J., Gonzalez-Reiche, A. S., Carreno, J. M., Hirsch, E., Tan, J., Khan, Z., Kriti, D., Ly, T., Kranitzky, B. et al. (2021) Real-Time Investigation of a Large Nosocomial Influenza A Outbreak Informed by Genomic Epidemiology. Clin Infect Dis, 73, e4375-e4383.
17. Gonzalez-Reiche, A. S., Hernandez, M. M., Sullivan, M. J., Ciferri, B., Alshammary, H., Obla, A., Fabre, S., Kleiner, G., Polanco, J., Khan, Z. et al. (2020) Introductions and early spread of SARS-CoV-2 in the New York City area. Science, 369, 297-301.
18. Lowe, T. M. and Chan, P. P. (2016) tRNAscan-SE On-line: integrating search and context for analysis of transfer RNA genes. Nucleic Acids Res, 44, W54-57.
19. Danecek, P., Bonfield, J. K., Liddle, J., Marshall, J., Ohan, V., Pollard, M. O., Whitwham, A., Keane, T., McCarthy, S. A., Davies, R. M. et al. (2021) Twelve years of SAMtools and BCFtools. Gigascience, 10, giab008.
20. Pedregosa, F., Varoquaux, G., Gramfort, A., Michel, V., Thirion, B., Grisel, O., Blondel, M., Prettenhofer, P., Weiss, R., Dubourg, V. et al. (2011) Scikit-learn: Machine Learning in Python. J Mach Learn Res, 12, 2825-2830.
21. McKinney, W. (2010) Data structures for statistical computing in python. Proceedings of the 9th Python in Science Conference 445, 51-56.
22. Harris, C. R., Millman, K. J., van der Walt, S. J., Gommers, R., Virtanen, P., Cournapeau, D., Wieser, E., Taylor, J., Berg, S., Smith, N.J. et al. (2020) Array programming with NumPy. Nature, 585, 357-362.
23. Hunter, J. D. (2007) Matplotlib: A 2D graphics environment. Comput Sci Eng, 9, 90-95.
24. H, W. (2016) ggplot2: Elegant Graphics for Data Analysis. Springer-Verlag, New York.
25. Helm, M. and Motorin, Y. (2017) Detecting RNA modifications in the epitranscriptome: predict and validate. Nat Rev Genet, 18, 275-291.
26. Clark, W. C., Evans, M. E., Dominissini, D., Zheng, G. and Pan, T. (2016) tRNA base methylation identification and quantification via high-throughput sequencing. RNA, 22, 1771-1784.
27. Cozen, A. E., Quartley, E., Holmes, A. D., Hrabeta-Robinson, E., Phizicky, E. M. and Lowe, T. M. (2015) ARM-seq: AlkB-facilitated RNA methylation sequencing reveals a complex landscape of modified tRNA fragments. Nat Methods, 12, 879-884.
28. Amunts, A., Brown, A., Toots, J., Scheres, S. H. W. and Ramakrishnan, V. (2015) Ribosome. The structure of the human mitochondrial ribosome. Science, 348, 95-98.

What is claimed is:

1. A sample collection method comprising:
assaying RNA, wherein the assaying comprises sequencing the RNA to obtain tRNA sequences and wherein the RNA is isolated from a nasopharyngeal sample from a subject having or suspected of having a SARS-CoV-2 infection.

2. The method of claim 1, wherein the assaying comprises obtaining a tRNA profile of the sample.

3. The method of claim 1 or 2, where the assaying comprises performing template ligation qPCR on the RNA.

4. The method of claim 2 or 3, wherein the tRNA profile comprises abundance, fragmentation, and modification information for at least 2 tRNAs.

5. The method of claim 2 or 3, wherein the tRNA profile comprises abundance, fragmentation, and modification information for at least 10 tRNAs.

6. The method of claim 2 or 3, wherein the tRNA profile comprises abundance, fragmentation, and modification information for at least 100 tRNAs.

7. The method of any one of claims 1-6, wherein the assaying comprises multiplex small RNA sequencing.

8. The method of any one of claims 1-7, wherein the tRNA sequences comprise sequences of one or more tRNAs listed in Table 1 or Table 2.

9. The method of any one of claims 1-8, wherein the tRNA sequences comprise sequences of one or more of the tRNA isoacceptor families AlaAGC, ArgACG, ProAGG, and GlnCTG.

10. The method of claim 9, wherein the method comprises determining a fragmentation profile of the one or more tRNA isoacceptor families.

11. The method of any one of claims 1-10, wherein the tRNA sequences comprise sequences of one or more of tRNAs chr1.trna32-MetiCAT, chr1.trna52-ProCGG, chr1.trna34-LeuCGG, and chr2.trna3-AlaAGC.

12. The method of claim 11, wherein the method comprises determining an abundance of the one or more tRNAs relative to ribosomal RNA in the sample.

13. The method of any one of claims 1-12, wherein the tRNA sequences comprise tRNA modification information for one or more of tRNA modifications mtValTAC.m¹A9, chr1.trna54-LysTTT.m¹A58, chr1.trna59-GluCTC.m¹A57, and chr1.tRNA69-AspGTC.m¹A9.

14. The method of claim 13, wherein the method comprises determining a frequency of the one or more tRNA modifications.

15. The method of any one of claims 1-14, wherein the method comprises determining a fragmentation profile of a tRNA of isoacceptor family ArgAGC, an abundance of tRNA chr1.tRNA32-MetiCAT, and modification information for tRNA modification mtValTAC.m¹A9.

16. The method of any one of claims 1-15, wherein the viral infection is an influenza infection.

17. The method of claim 16, wherein the viral infection is a coronavirus infection.

18. The method of claim 17 wherein the coronavirus infection is a SARS-CoV-2 infection.

19. The method of claim 18, further comprising identifying the subject as having severe COVID-19 based on analysis of the tRNA sequences.

20. The method of claim 18, further comprising identifying the subject as having mild COVID-19 based on analysis of the tRNA sequences.

21. The method of claim 18, further comprising diagnosing the subject as having COVID-19.

22. The method of any one of claims 18-21, further comprising administering to the subject a COVID-19 therapy.

23. The method of claim 22, wherein the COVID-19 therapy comprises an antibody therapeutic, an antiviral therapeutic, or convalescent plasma.

24. A method of treating a SARS-CoV-2 infection in a subject, the method comprising:
assaying RNA, wherein the assaying comprises sequencing the RNA to obtain tRNA sequences, and wherein the RNA is isolated from a nasopharyngeal sample from a subject having or suspected of having a SARS-CoV-2 infection; and
administering to the subject a therapeutically effective amount of a COVID-19 therapy.

25. The method of claim 24, wherein the assaying comprises obtaining a tRNA profile of the sample.

26. The method of claim 24 or 25, where the assaying comprises performing template ligation qPCR on the RNA.

27. The method of claim 25 or 26, wherein the tRNA profile comprises abundance, fragmentation, and modification information for at least 2 tRNAs.

28. The method of claim 25 or 26, wherein the tRNA profile comprises abundance, fragmentation, and modification information for at least 10 tRNAs.

29. The method of claim 25 or 26, wherein the tRNA profile comprises abundance, fragmentation, and modification information for at least 100 tRNAs.

30. The method of any one of claims 24-29, wherein the assaying comprises multiplex small RNA sequencing.

31. The method of any one of claims 24-30, wherein the tRNA sequences comprise sequences of one or more tRNAs listed in Table 1 or Table 2.

32. The method of any one of claims 24-31, wherein the tRNA sequences comprise sequences of one or more of the tRNA isoacceptor families AlaAGC, ArgACG, ProAGG, and GlnCTG.

33. The method of claim 32, wherein the method comprises determining a fragmentation profile of the one or more tRNA isoacceptor families.

34. The method of any one of claims 24-33, wherein the tRNA sequences comprise sequences of one or more of tRNAs chr1.trna32-MetCAT, chr1.trna52-ProCGG, chr1.trna34-LeuCGG, and chr2.trna3-AlaAGC.

35. The method of claim 34, wherein the method comprises determining an abundance of the one or more tRNAs relative to ribosomal RNA in the sample.

36. The method of any one of claims 24-35, wherein the tRNA sequences comprise tRNA modification information for one or more of tRNA modifications mtValTAC.m¹A9, chr1.trna54-LysTTT.m¹A58, chr1.trna59-GluCTC.m¹A57, and chr1.tRNA69-AspGTC.m¹A9.

37. The method of claim 36, wherein the method comprises determining a frequency of the one or more tRNA modifications.

38. The method of any one of claims 25-37, wherein the method comprises determining a fragmentation profile of a tRNA of isoacceptor family ArgAGC, an abundance of tRNA chr1.tRNA32-MetiCAT, and modification information for tRNA modification mtValTAC.m¹A9.

39. The method of any one of claims 24-38, wherein the COVID-19 therapy comprises an antibody therapeutic, an antiviral therapeutic, or convalescent plasma.

40. A method for diagnosing or prognosing a subject with COVID-19, the method comprising:
assaying RNA, wherein the assaying comprises sequencing the RNA to obtain tRNA sequences; and
analyzing the tRNA sequences to identify the subject as having mild COVID-19 or severe COVID-19,
wherein the RNA is isolated from a nasopharyngeal sample from a subject having or suspected of having a SARS-CoV-2 infection.

41. The method of claim 40, wherein the assaying comprises obtaining a tRNA profile of the sample.

42. The method of claim 24 or 25, where the assaying comprises performing template ligation qPCR on the RNA.

43. The method of claim 41 or 42, wherein the tRNA profile comprises abundance, fragmentation, and modification information for at least 2 tRNAs.

44. The method of claim 41 or 42, wherein the tRNA profile comprises abundance, fragmentation, and modification information for at least 10 tRNAs.

45. The method of claim 41 or 42, wherein the tRNA profile comprises abundance, fragmentation, and modification information for at least 100 tRNAs.

46. The method of any one of claims 40-45, wherein the assaying comprises multiplex small RNA sequencing.

47. The method of any one of claims 40-46, wherein the tRNA sequences comprise sequences of one or more tRNAs listed in Table 1 or Table 2.

48. The method of any one of claims 40-47, wherein the tRNA sequences comprise sequences of one or more of the tRNA isoacceptor families AlaAGC, ArgACG, ProAGG, and GlnCTG.

49. The method of claim 48, wherein the method comprises determining a fragmentation profile of the one or more tRNA isoacceptor families.

50. The method of any one of claims 40-49, wherein the tRNA sequences comprise sequences of one or more of tRNAs chr1.trna32-MetiCAT, chr1.trna52-ProCGG, chr1.trna34-LeuCGG, and chr2.trna3-AlaAGC.

51. The method of claim 50, wherein the method comprises determining an abundance of the one or more tRNAs relative to ribosomal RNA in the sample.

52. The method of any one of claims 40-51, wherein the tRNA sequences comprise tRNA modification information for one or more of tRNA modifications mtValTAC.m¹A9, chr1.trna54-LysTTT.m¹A58, chr1.trna59-GluCTC.m¹A57, and chr1.tRNA69-AspGTC.m¹A9.

53. The method of claim 52, wherein the method comprises determining a frequency of the one or more tRNA modifications.

54. The method of any one of claims 40-53, wherein the method comprises determining a fragmentation profile of a tRNA of isoacceptor family ArgAGC, an abundance of tRNA chr1.tRNA32-MetiCAT, and modification information for tRNA modification mtValTAC.m¹A9.

55. The method of any one of claims 40-54, wherein, in (c), the subject is identified as having mild COVID-19.

56. The method of any one of claims 40-55, wherein, in (c), the subject is identified as having severe COVID-19.

57. The method of any one of claims 40-56, further comprising administering to the subject a COVID-19 therapy.

58. The method of claim 57, wherein the COVID-19 therapy comprises an antibody therapeutic, an antiviral therapeutic, or convalescent plasma.

59. A method for evaluating a subject having or suspected of having a SARS-CoV-2 infection, the method comprising:
assaying RNA to obtain a tRNA profile comprising abundance, fragmentation, and modification information for at least 2 tRNAs of Table 1 or Table 2, wherein the RNA is isolated from a nasopharyngeal sample from a subject having or suspected of having a SARS-CoV-2 infection.

60. The method of claim 59, wherein the tRNA profile comprises abundance, fragmentation, and modification information for at least 10 tRNAs of Table 1 or Table 2.

61. The method of claim 59 or 60, wherein the assaying comprises multiplex small RNA sequencing.

62. The method of any one of claims 59-61, wherein the assaying comprises performing template ligation qPCR on the RNA.

63. The method of any one of claims 59-62, wherein the tRNA sequences comprise sequences of one or more of the tRNA isoacceptor families AlaAGC, ArgACG, ProAGG, and GlnCTG.

64. The method of claim 63, wherein the method comprises determining a fragmentation profile of the one or more tRNA isoacceptor families.

65. The method of any one of claims 59-64, wherein the tRNA sequences comprise sequences of one or more of tRNAs chr1.trna32-MetiCAT, chr1.trna52-ProCGG, chr1.trna34-LeuCGG, and chr2.trna3-AlaAGC.

66. The method of claim 65, wherein the method comprises determining an abundance of the one or more tRNAs relative to ribosomal RNA in the sample.

67. The method of any one of claims 59-66, wherein the tRNA sequences comprise tRNA modification information for one or more of tRNA modifications mtValTAC.m¹A9, chr1.trna54-LysTTT.m¹A58, chr1.trna59-GluCTC.m¹A57 and chr1.tRNA69-AspGTC.m¹A9.

68. The method of claim 67, wherein the method comprises determining a frequency of the one or more tRNA modifications.

69. The method of any one of claims 59-68, wherein the method comprises determining a fragmentation profile of a tRNA of isoacceptor family ArgAGC, an abundance of tRNA chr1.tRNA32-MetiCAT, and modification information for tRNA modification mtValTAC.m1A9.

70. A method of quantitatively detecting N1-methyladenosine (m¹A) at a site of interest on a tRNA in a sample, the method comprising:
binding a first oligonucleotide to the tRNA in the sample, wherein the first oligonucleotide:
(i) is complementary to the tRNA both at the site of interest and upstream of the site of interest; and
(ii) comprises a phosphorylated thymine at its 5′-terminus;
binding a second oligonucleotide to the tRNA in the sample, wherein the second oligonucleotide is complementary to the tRNA directly downstream of the site of interest;
contacting the first oligonucleotide and second oligonucleotide with a ligase to generate a ligated product; and
assaying the sample to quantitatively detect an amount of ligated product.

71. The method of claim 70, wherein first oligonucleotide and second oligonucleotide are DNA oligonucleotides.

72. The method of claim 70 or 71, wherein the ligase comprises a SplintR ligase.

73. The method of any one of claims 71-72, wherein the ligated product is not generated when the site of interest comprises an m¹A modification.

74. The method of any one of claims 71-73, wherein the first oligonucleotide is 10, 11, 12, 13, 14, 15, 16, 17, 18, 19, or 20 nucleotides in length.

75. The method of any one of claims 71-74, wherein the second oligonucleotide is 10, 11, 12, 13, 14, 15, 16, 17, 18, 19, or 20 nucleotides in length.

76. The method of any one of claims 71-75, wherein the tRNA comprises at least 1 tRNAs of Table 1 or Table 2.

77. The method of any one of claims 71-76, further comprising
binding a third oligonucleotide and fourth oligonucleotide to a second site on the tRNA, wherein the second site is different from the site of interest and does not comprise a known m¹A modification site and wherein the third oligonucleotide binds directly adjacent to the fourth oligonucleotide;
contacting the third oligonucleotide and fourth oligonucleotide with a ligase to generate a second ligated product; and
assaying the sample to quantitatively detect an amount of the second ligated product.

78. The method of claim 77, further comprising comparing the amount of the ligated product to the amount of the second ligated product.

79. The method of any one of claims 71-78, wherein the analyzing comprises quantitative PCR.

80. The method of any one of claims 71-79, wherein the sample comprises a nasopharyngeal sample.

81. The method of any one of claims 71-80, wherein the sample comprises RNA isolated from a nasopharyngeal sample.


SEQUENCE LISTING (26 sequences)

SEQ ID NO: 1 - DNA, 15 bp, synthetic construct
  source 1..15: mol_type = other DNA
tccagctacctagct

SEQ ID NO: 2 - RNA, 33 nt, synthetic construct
  source 1..33: mol_type = other RNA
aggtcgatggatcgaaaccatcctctgctacca

SEQ ID NO: 3 - RNA, 33 nt, synthetic construct; identical to SEQ ID NO: 2
  source 1..33: mol_type = other RNA
  modified_base 15: mod_base = m1a

SEQ ID NO: 4 - DNA, 15 bp, synthetic construct
  source 1..15: mol_type = other DNA
  modified_base 1: mod_base = OTHER; note = HEX labeled probe
tagcagaggatggtt

SEQ ID NO: 5 - DNA, 15 bp, synthetic construct
  source 1..15: mol_type = other DNA
gacgcacctccagct

SEQ ID NO: 6 - RNA, 43 nt, synthetic construct
  source 1..43: mol_type = other RNA
  modified_base 18: mod_base = m1a
ggtctgcgtggaggtcgacgactccgtcgcagactacggacct

SEQ ID NO: 7 - RNA, 43 nt, synthetic construct; identical to SEQ ID NO: 6
  source 1..43: mol_type = other RNA

SEQ ID NO: 8 - DNA, 15 bp, synthetic construct
  source 1..15: mol_type = other DNA
  modified_base 1: mod_base = OTHER; note = Cy5 labeled probe
tctgcgacggagtcg

SEQ ID NO: 9 - DNA, 11 bp, synthetic construct
  source 1..11: mol_type = other DNA
  modified_base 1: mod_base = OTHER; note = Cy5 labeled probe
tggtccgtagt

SEQ ID NO: 10 - DNA, 15 bp, synthetic construct
  source 1..15: mol_type = other DNA
agacgcacctccagc

SEQ ID NO: 11 - DNA, 11 bp, synthetic construct
  source 1..11: mol_type = other DNA
  modified_base 1: mod_base = OTHER; note = Cy5 labeled probe
tggtccgtagt

SEQ ID NO: 12 - DNA, 18 bp, synthetic construct
  source 1..18: mol_type = other DNA
cctaaatcgagtcaaccc

SEQ ID NO: 13 - DNA, 17 bp, synthetic construct
  source 1..17: mol_type = other DNA
cctccaggacacaagct

SEQ ID NO: 14 - RNA, 76 nt, synthetic construct
  source 1..76: mol_type = other RNA
  modified_base 58: mod_base = m1a
gcggatttagctcagttgggagagcgccagactgaagatctggaggtcctgtgttcgatccacagaattcgcacca

SEQ ID NO: 15 - DNA, 15 bp, synthetic construct
  source 1..15: mol_type = other DNA
  modified_base 1: mod_base = OTHER; note = Texas Red labeled probe
tcagtctggcgctct

SEQ ID NO: 16 - DNA, 16 bp, synthetic construct
  source 1..16: mol_type = other DNA
  modified_base 1: mod_base = OTHER; note = Cy5 labeled probe
gtgcgaattctgtgga

SEQ ID NO: 17 - RNA, 38 nt, synthetic construct
  source 1..38: mol_type = other RNA
cccagaggtcgatggatcgaaaccatcctctgctacca

SEQ ID NO: 18 - DNA, 15 bp, synthetic construct
  source 1..15: mol_type = other DNA
tagcagaggatggtt

SEQ ID NO: 19 - DNA, 15 bp, synthetic construct
  source 1..15: mol_type = other DNA
cgttggagccaagct

SEQ ID NO: 20 - RNA, 39 nt, synthetic construct
  source 1..39: mol_type = other RNA
  modified_base 21: mod_base = m1a
gccgcagcaacctcggttcgaatccgagtcacggcacca

SEQ ID NO: 21 - DNA, 15 bp, synthetic construct
  source 1..15: mol_type = other DNA
  modified_base 1: mod_base = OTHER; note = Texas Red labeled probe
tgccgtgactcggat

SEQ ID NO: 22 - DNA, 15 bp, synthetic construct
  source 1..15: mol_type = other DNA
ttaccatcgcgcaga

SEQ ID NO: 23 - RNA, 39 nt, synthetic construct
  source 1..39: mol_type = other RNA
ggcgcaatggtagcgcgtctgactccagatcagaaggtt

SEQ ID NO: 24 - DNA, 15 bp, synthetic construct
  source 1..15: mol_type = other DNA
  modified_base 1: mod_base = OTHER; note = FAM labeled probe
ttctgatctggagtc

SEQ ID NO: 25 - RNA, 30 nt, synthetic construct
  source 1..30: mol_type = other RNA
  modified_base 15: mod_base = m1a
aggtcgatggatcgaaaccatcctctgcta

SEQ ID NO: 26 - DNA, 10 bp, synthetic construct
  source 1..10: mol_type = other DNA
tgctgaggca